(19) United States
(12) Patent Application Publication
(10) Pub. No.: US 2007/0276203 A1
(43) Pub. Date: Nov. 29, 2007

(54) Method and System for Improving Health Status of Members of an Entity

(76) Inventor: Aaron Day, Boston, MA (US)

Correspondence Address: BROMBERG & SUNSTEIN LLP, 125 SUMMER STREET, BOSTON, MA, 02110-1618, US

(21) Appl. No.: 11/672,667

(22) Filed: Feb. 8, 2007

Related U.S. Application Data
(63) Continuation-in-part of application No. 11/105,956, filed on Apr. 14, 2005.
(60) Provisional application No. 60/771,347, filed on Feb. 8, 2006.

(51) Int. Cl. A61B 5/00

(52) U.S. Cl. 600/301

(57) ABSTRACT

A method is provided for improving the health status of members of an entity by using a program in which members may enroll. The method includes publicizing to members a pool of rewards available to those who enroll in the program. For each enrolled member, a target physiological condition is established and successive physiological measurements related to the condition are obtained over a period of time. The target physiological condition and the successive physiological measurements are stored in a digital computer system and a computer process is used to determine and store a score of such member's proximity to the target physiological condition based on each such measurement. An award from the pool to one or more enrolled members is granted based on their scores at the end of the period.

### CROSS-REFERENCE TO RELATED APPLICATIONS

This patent application claims priority from U.S. Provisional Patent Application No. 60/771,347 filed Feb. 8, 2006 and is also a continuation in part of pending U.S. patent application Ser. No. 11/105,956 filed Apr. 14, 2005. These related patent applications are hereby incorporated herein by reference.

### TECHNICAL FIELD AND BACKGROUND ART

The present invention relates to a system for improving the health status of members in an entity. Employers are facing a healthcare crisis in that obesity has become a health epidemic for employees. Health insurance costs have grown by double digits every year for the past 5 years. Health Affairs estimates that 27% of the increase in healthcare costs from 1987-2001 can be attributed to the rise in obesity. According to the Employee Benefit Research Institute, based on the current growth in insurance costs, up to 67% of an employee's total compensation could be in the form of insurance benefits within 10 years

Most large corporations self-insure which means that employee claims are a direct cost. As a result, corporations are moving toward consumer-driven care as a way to drive down costs. The National Business Group on Health (NBGH) is a national health-issue focused non-profit organization. The NBGH represents over 200 members (primarily Fortune 500 companies who provide health coverage for over 45 million US workers, retirees, and their families). In 2003, the NBGH launched the Institute on the Costs and Health Effects of Obesity to produce tools to estimate the cost of obesity to employers and to initiate employee communication regarding the obesity problem. Employers identified the need for a weight loss and management vendor that involves minimal administration, is fully HIPAA compliant, reduces medical care costs, increases employee productivity, and produces a solid return-on-investment (ROI).

It is known in the prior art to provide rewards for self-reported behavior. For example, frequent flyer programs come in two varieties, simple incentive structures and pyramiding incentives. With simple incentives the number of miles a person flies determines how many points they receive. With Pyramiding incentives, a person gets additional privileges as they fly more miles (free upgrades, free club memberships).

Further, major incentive companies like Maritz, BI (Business Incentives), Carlson Companies, Incentone, and a myriad of other companies are known as the premium incentive businesses. All of their programs are structured like a frequent-flyer program. The objective of these programs is to change the behavior of a participating group in the direction that the company wants it to go. There are simple equations to determine how much incentive is required to produce changes in behavior. The types of goals that companies usually pursue are sales and marketing programs to increase sales (either dollar volume or units), to push a low performing product through the channel, to extend product life cycles, to change product mix, introduce a new product, build customer loyalty (or dealer loyalty) increase performance in an operational area (i.e. claims or check processing, or manufacturing productivity, to reduce costs, retention, attendance, safety, wellness, ideas, or any other dimension of human behavior within the control of the participant group). Destiny Health is an attempt to encourage employees to behave in ways that promote health. It offers merchandise rewards and travel rewards for this participation.

### SUMMARY OF THE INVENTION

In a first embodiment of the invention there is provided a method for improving the health status of members of an entity by using a program in which members may enroll. The method includes publicizing to members a pool of rewards available to those who enroll in the program. For each enrolled member, a target physiological condition is established and successive physiological measurements related to the condition are obtained over a period of time. The target physiological condition and the successive physiological measurements are stored in a digital computer system and a computer process is used to determine and store a score of such member's proximity to the target physiological condition based on each such measurement. An award from the pool to one or more enrolled members is granted based on their scores at the end of the period.

In accordance with a related embodiment, at least some of the measurements may be collected using a small, wearable device. At least some of the measurements may be provided to the computer system via a cellular telephone. The value of the set of awards granted may be determined at least in part based on calculated reduction in presumed cost associated with the enrolled members as a result of their changes in physiological condition over the period. In accordance with yet another related embodiment, the value of a given award granted to a given enrolled member may be determined at least in part based on calculated reduction in presumed cost associated with the given member's change in physiological condition over the period.

In accordance with a further related embodiment, establishing a target physiological condition and obtaining successive physiological measurements may be performed under the auspices of a party distinct from the entity. In accordance with another related embodiment, the target physiological condition and the successive physiological measurements may be kept confidential from the entity. In accordance with a further related embodiment, the method may also include using a computer process to determine which enrolled members are granted an award based on their scores.

In accordance with additional related embodiments, the target physiological condition and the successive physiological measurements may include at least one or a combination of at least two of: the member's weight, blood pressure, body mass index, pulse rate, glycemic index, cholesterol level, levels of expression of designated genes, and a ratio of body composition to fat percentage.

In accordance with yet another related embodiment, the entity may include a business entity and the member may be an employee of the business entity.

In accordance with another embodiment of the invention, there may exist a plurality of groups of enrolled members and the method may include determining a collective score for each of the plurality of groups based on the scores of enrolled members in each group, and granting an award from the pool to one or more of the groups based on the scores of the groups. In accordance with yet another related embodiment, the entity may be a collection of organizations and each group may be a distinct set of one or more organizations. In accordance with a further related embodiment, granting the award may be performed under the auspices of the party distinct from the entity. The value of the set of awards granted may be determined at least in part based on calculated reduction in presumed cost associated with the enrolled members as a result of their changes in physiological condition over the period. Further, the value of a given award granted to a given enrolled member may be determined at least in part based on calculated reduction in presumed cost associated with the given member's change in physiological condition over the period.

Yet another embodiment of the invention provides a method for improving the health status of members of an entity by using a program in which members may enroll. The method includes notifying the members of a pool of rewards available to those who enroll in the program. Also, for each enrolled member, the method includes establishing a target physiological condition, and obtaining successive physiological measurements related to the condition over a period of time, and storing in a digital computer system the target physiological condition and the successive physiological measurements, wherein such measurements are obtained under personal supervision at a set of locations remote from the digital computer system and are provided to the computer system over a network, wherein at least some of the locations are under the control of an organization that is a measurement partner of the program; and using a computer process to determine and store a score of such member's proximity to the target physiological condition based on each such measurement. Finally the method includes granting an award from the pool to one or more enrolled members based on their scores at the end of the period.

In various related embodiments, at least some of such measurements are provided to the computer system via entries made in web pages over the network. Alternatively, or in addition, the measurement partner is enrolled in the program by a process using web-page data entry over the network. Also alternatively or in addition, the entity is enrolled in the program by using web-page data entry, so as to be able to offer enrollment to members of the entity, In a case wherein there exists a plurality of groups (which we call “teams” below) of enrolled members, the method may include determining a collective score for each of the plurality of groups based on the scores of enrolled members in each group, and granting an award from the pool to one or more of the groups based on the scores of the groups.

In another related embodiment, the entity is a collection of organizations and each group is a distinct set of one or more organizations. Alternatively or in addition, storing in a digital computer system the target physiological condition and the successive physiological measurements includes storing a combination of at least two of: the member's weight, blood pressure, body mass index, pulse rate, glycemic index, cholesterol level, levels of expression of designated genes, and ratio of body composition to fat percentage. Alternatively or in addition, the entity includes a business entity, and the member is an employee of the business entity.

Another similar embodiment provides a method for improving the health status of members of an entity by using a program in which members may enroll, and the method includes notifying the members of a pool of rewards available to those who enroll in the program. The method also includes for a set including at least some of the members who enroll, causing each member of the set to be associated with one of a plurality of teams. Also, for each enrolled member, the method includes establishing a target physiological condition, and obtaining successive physiological measurements related to the condition over a period of time, storing in a digital computer system the target physiological condition and the successive physiological measurements, wherein such measurements are obtained at a set of locations remote from the digital computer system and are provided to the computer system over a network, wherein at least some of the locations are under the control of an organization that is a measurement partner of the program and wherein the measurement partner is enrolled in the program by a process using web-page data entry over the network; and using a computer process to determine and store a score of such member's proximity to the target physiological condition based on each such measurement. Finally, the method includes determining a collective score for each of the plurality of teams based on the scores of enrolled members on each team, and granting an award from the pool to one or more of the teams based on the scores of the teams.

In a related embodiment, causing each member of the set to be associated with one of a plurality of teams includes permitting each such member to voluntarily join one of the teams. Alternatively or in addition, the entity is a collection of organizations, and causing each member of the set to be associated with one of a plurality of teams includes establishing a separate team for each organization, each team comprised of enrolled members from a single organization of the collection, so that the organizations compete with one another as teams. Also alternatively or in addition, for each enrolled member, the method includes making available to such member over a network in communication with the digital computer, information showing the member's progress toward such member's physiological target and, if applicable, competitive performance data for a team with respect to which such member is associated. Causing each member of the set to be associated with one of a plurality of teams may include balancing the teams so that an average of one or more characteristics of the members in each team is approximately equal. Balancing the teams may include calculating a body mass index for each member of the set and selecting each team so that the average body mass index for each team is approximately equal.

In yet another embodiment, there is provided a method for improving the health status of members of an entity by using a program in which members may enroll, and the method includes notifying the members of a pool of rewards available to those who enroll in the program. For each enrolled member, the method includes establishing a target physiological condition, and obtaining successive physiological measurements related to the condition over a period of time, storing in a digital computer system the target physiological condition and the successive physiological measurements, and using a computer process to determine and store a score of such member's proximity to the target physiological condition based on each such measurement. The method also includes using stored data aggregated with respect to a selected class of enrolled members to identify a strategy successfully employed by members of the class to facilitate achieving their respective target physiological conditions, and communicating over a network in communication with the digital computer system a recommendation of the strategy to relevant enrolled members. Finally, the method includes granting an award from the pool to one or more enrolled members based on their scores at the end of the period.

Another embodiment of the invention similarly provides method for improving the health status of members of an entity by using a program in which members may enroll, and the method includes notifying the members a pool of rewards available to those who enroll in the program. The method also includes, for each enrolled member, establishing a target physiological condition, and obtaining successive physiological measurements related to the condition over a period of time, storing in a digital computer system the target physiological condition and the successive physiological measurements, and using a computer process to determine and store a score of such member's proximity to the target physiological condition based on each such measurement; communicating over the network to such enrolled member such member's progress toward the target physiological condition and, optionally, such member's behavior, and also optionally advertisements that are deemed pertinent to such member based on data pertinent to such member. Finally the method includes granting an award from the pool to one or more enrolled members based on their scores at the end of the period.

In related embodiments, obtaining successive physiological measurements may include collecting the measurements using a small, wearable device. The small, wearable device may include a pedometer, a GPS device and/or a physiological sensor. Obtaining successive physiological measurements may include providing the measurements via a cellular telephone.

In another embodiment, a method for improving the health status of a member enrolled in a program, the program providing a pool of rewards available to the member, includes determining what health resources are offered to the members enrolled in the program, identifying, among the health resources offered, a selected group of health resources utilized by a subset of the members who have had success in achieving targeted physiological conditions and identifying a set of characteristics for each member of the subset. The method also includes correlating the set of characteristics for each member of the subset with each health resource in the selected group for each physiological condition and providing a recommendation of one or more health resources to the member based on the correlation and the set of characteristics of the member enrolled.

In related embodiments, the set of characteristics for each member may include at least one characteristic selected from the group including gender, age, weight, and geographic location. The method may further include providing user recommendations of health resources that the members of the subset have had success in using to achieve targeted physiological conditions, so that correlating the identifying characteristics for each member of the subset with each health resource in the selected group includes the identified selected group of health resources and the user recommendation of health resources. Determining what health resources are offered to the members enrolled in the program may include determining what to charge the health resource to be included as a resource based on the success of the members in achieving their targeted physiological conditions using the health resource.

### BRIEF DESCRIPTION OF THE DRAWINGS

The foregoing features of the invention will be more readily understood by reference to the following detailed description, taken with reference to the accompanying drawings, in which:

FIG. 1 is a flow chart illustrating a system for improving the health status of a group or member of an entity;

FIG. 2 is a block diagram illustrating a software architecture diagram for the system of FIG. 1;

FIG. 3 is a flow chart illustrating a system for establishing a pool of rewards;

FIG. 4 is a graphical illustration showing the change in weight for a member over a three year time period;

FIG. 5 is a graphical illustration showing the distribution in savings from the system based on a certain pool of rewards and distribution to a third party for managing the system;

FIG. 6 is a flow chart illustrating a process for a member enrolling in the system;

FIG. 7 is a screen shot design for a log-in page for a website tracking tool for a member;

FIG. 8 is a screen shot design for a member registering in the program through a website;

FIG. 9 is a screen shot design for a member providing authentication as to their individual identity;

FIG. 10 is a screen shot design for a registration confirmation page;

FIG. 11 is a flow chart illustrating the process of measuring a member's physiological condition;

FIG. 12 is a screen shot design for a website log-in page for an application that records members' physiological condition and stores them in a database;

FIG. 13 is a screen shot design for a website scheduling page that allows an entity to coordinate the logistics of measuring the physiological condition of members of an entity;

FIG. 14 is a screen shot design for a website page used for recording the specific member's physiological condition;

FIG. 15 is a screen shot design for a website page that confirms the member's current physiological condition and provides a physiological target score for a specified time period;

FIG. 16 is a screen shot design for an e-mail confirmation for a member that has signed up for the program.

FIG. 17 is a screen shot design of a log-in page for a tracking tool that allows a member to monitor their physiological condition;

FIG. 18 is a screen shot design for a website page that allows a member to track their caloric intake and caloric expenditure against their individual goal;

FIG. 19 is a screen shot design for a website page that allows a member to monitor their progress against their goal;

FIG. 20 is a screen shot design for a website page that allows a member to communicate and interact with other participants in the system;

FIG. 21 is a screen shot design for a website page that allows an entity to view the aggregate success of their members;

FIG. 22 is a screen shot design for a website page that allows an entity to view their financial return for the program factoring in program costs and the distribution of the pool of rewards;

FIG. 23 is a screen shot design for a web page that allows an entity to view their overall breakeven analysis for the program period;

FIG. 24 is a block diagram illustrating logical flow for entity enrollment in a program in accordance with an embodiment of the present invention;

FIG. 25 is a representation of a web page presented to a representative of an entity for enrolling in the program of FIG. 24;

FIGS. 26, 28, 29 and 30 are representations of web pages presented to a representative of an entity for configuring the program of FIG. 24;

FIG. 27 is a representation of a web page presented to a representative of an entity for handling publication of the program of FIG. 24;

FIG. 31 is a block diagram, in accordance with an embodiment of the present invention, illustrating logical flow associated with user enrollment in the program of FIG. 24;

FIG. 32 is a representation of a web page, pursuant to the logical flow of FIG. 31, presented to a user enrolling in the program of FIG. 24 for handling verification of identification;

FIG. 33 is a representation of a web page, in accordance with an embodiment of the present invention, presented to a user enrolling in the program of FIG. 24 for handling weight measurement options;

FIG. 34 is a representation of a web page, in accordance with an embodiment of the present invention, presented to a user enrolling in the program of FIG. 24 for handling communication and election of team participation;

FIG. 35 is a representation of a web page, in accordance with an embodiment of the present invention, presented to a user enrolling in the program of FIG. 24 for handling team participation options;

FIG. 36 is a representation of a web page, in accordance with an embodiment of the present invention, presented to a user confirming the user's enrollment in the program of FIG. 24;

FIG. 37 is block diagram of logical flow, in accordance with an embodiment of the present invention, associated with enrollment of a partner in the program of FIG. 24;

FIG. 38 is a representation of a web page, in accordance with an embodiment of the present invention, presented to an organization representative for enrollment of the organization as a partner in the program of FIG. 24;

FIG. 39 is a representation of a web page, in accordance with an embodiment of the present invention, presented to the organization representative for providing data pertinent to operating as a measurement partner in the program of FIG. 24;

FIG. 40 is a representation of a web page, in accordance with an embodiment of the present invention, presented to the organization representative for training and certification pertinent to operating as a measurement partner in the program of FIG. 24;

FIG. 41 is a representation of a web page, in accordance with an embodiment of the present invention, presented to the organization representative for publicizing to subsidiary locations of the organization the fact that the organization has become a partner in the program of FIG. 24;

FIG. 42 is a block diagram showing a process, in accordance with an embodiment of the present invention, by which staff of a measurement partner obtains measurements of a user and provides them using web pages over a network to a database;

FIG. 43 is a block diagram, in accordance with an embodiment of the present invention, showing processes by which a user obtains information concerning behavior and results of participation in the program of FIG. 24 and communicates with partners and others;

FIG. 44 is a representation of a web page, in accordance with an embodiment of the present invention, presented to the user for logging in to obtain the information of FIG. 43;

FIG. 45 is a representation of a web page, in accordance with an embodiment of the present invention, presented to the user for tracking results of participation in the program, viewing competitive data, communicating with teammates, and receiving advertising content;

FIG. 46 is a representation of a web page, in accordance with an embodiment of the present invention, presented to the user for tracking behavior and receiving advertising content;

FIG. 47 is a representation of a web page, in accordance with an embodiment of the present invention, presented to the user for viewing offerings by partners enrolled in the program of FIG. 24;

FIG. 48 is a representation of a web page, in accordance with an embodiment of the present invention, presented to the user for sharing messages (for example recipes) with other users;

FIG. 49 is a representation of a supportive e-mail message, in accordance with an embodiment of the present invention, to the user providing competitive and behavior data;

FIG. 50 is a representation of a merchandise web page, in accordance with an embodiment of the present invention, presented to the user for displaying merchandise that may be obtained using reward points obtained by participation in the program;

FIG. 51 is a block diagram showing how recommendations to users in the program are based on both pooled user data and on third party data;

FIG. 52 is a representation of a web page, in accordance with an embodiment of the present invention, presented to the user, for providing recommendations to the user based on aggregate data from other program users.

FIG. 53 is a representation of a web page, in accordance with an embodiment of the present invention, presented to the user, for showing foods and activities that may be selected, rated, and shared by the user among a community of users participating in the program;

FIG. 54 is a representation of a web page, in accordance with an embodiment of the present invention, presented to the user, for providing options, for selecting a partner, that may be customized for the user taking into account patterns of similar users;

FIG. 55 is a block diagram, in accordance with an embodiment of the present invention, showing potential sources of user rewards as coming not only from contributions by an entity that is an employer, for example, but also from partners participating in the program and also from other users;

FIG. 56 is a block diagram showing potential sources for collecting and/or entering a user's physiological measurements in accordance with an embodiment of the present invention;

FIG. 57 is block diagram of logical flow associated with how the measurements in FIG. 56 may be utilized in accordance with an embodiment of the present invention;

FIG. 58 is a block diagram of logical flow associated with the selection and recommendation of health resources offered to a user in accordance with an embodiment of the present invention;

FIG. 59 is a block diagram showing the process associated with the selection and recommendation of health resources offered to a user in accordance with an embodiment of the present invention; and

FIG. 60 is a block diagram of logical flow associated with grouping users into balanced teams in accordance with an embodiment of the present invention.

### DETAILED DESCRIPTION OF SPECIFIC EMBODIMENTS

Definitions. As used in this description and the accompanying claims, the following terms shall have the meanings indicated, unless the context otherwise requires:

Physiological condition means a condition of health of a subject as determined by measurement of one or more parameters indicative of health. Such parameters may include (but are not limited to) one or more of body mass index, blood pressure, pulse rate, glycemic index, cholesterol level, weight, levels of expression of designated genes, and the ratio of body composition to fat percentage.

A target physiological condition may include target values for one or more parameters associated with physiological condition: in other words, target values for one or more parameters such as a subject's (or member's) body mass index, blood pressure, pulse rate, glycemic index, cholesterol level, weight, levels of expression of designated genes, and the ratio of body composition to fat percentage. Current values of the pertinent parameter or parameters are determined from time to time by measurement, with a view to determining progress of the subject or member toward the target physiological condition.

In general, the invention described herein is directed to a method that improves the health status of members of an entity. A product blueprint illustrating a pilot program called “Realize” that incorporates features of the invention is attached hereto as Appendix A. Appendix A is hereby incorporated herein, in its entirety, by reference.

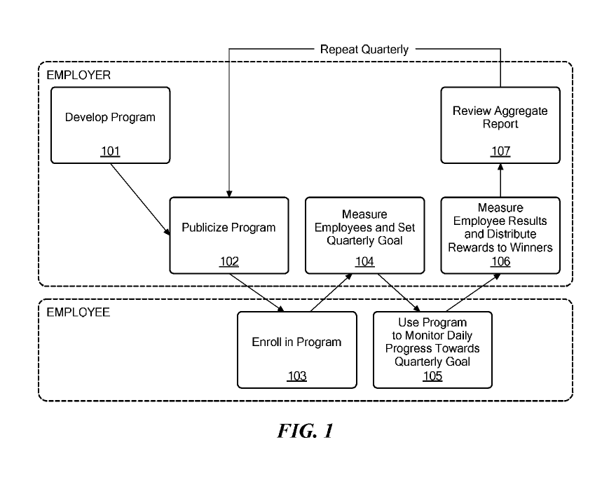

FIG. 1 is a flow chart illustrating a system for improving the health status of a group or member of an entity. In accordance with one aspect, the invention relates to a system for establishing and publishing a pool of rewards to members of an entity (such as a company, firm, partnership, or corporation), tracking the member or group of members, or the entity's physiological condition over time, and distributing rewards to the member, group of members, or entity for meeting specified wellness targets. In accordance with another aspect, the invention relates to a similar system wherein any one or more of tracking, publishing, and/or distribution of rewards is performed by party other than the member, group of members, or entity.

In process 101, a program is developed. Such development includes, but is not limited to, establishing a pool of rewards for distribution to members who attain a specific physiological condition during the program period. The program is publicized 102 in order to make members aware of the existence of the program via online (e.g., the intranet, e-mail) and offline (e.g., flyers, posters, mail, etc.) methods. One or more members or entities enroll in the program in process 103. Enrollment includes a registration process for participation in the program using an internet enabled computer system. A member's physiological condition is measured and quarterly goals are set in accordance with the members' wishes and with his or her physiological condition, in process 104. A member (such as an employee) or an entity (or its representative) may enter a member's initial physiological condition into a computer system and then have the computer system generate a quarterly target physiological condition for the member. (Optionally, the member's self-measurement of drug, alcohol, and/or cigarette consumption may be entered into the computer system as well.)

A member's daily progress toward his or her quarterly goal is monitored in process 105 (via, for example a computer program or any tracking tool that allows a member to track his or her behavior and change in physiological condition during the program period). Members' results are measured and rewards are distributed to winners in process 106. For example, an entity or its representative may be permitted to enter a member's final initial physiological condition into a computer system. The computer system may then compare the member's final physiological condition against his or her computer generated target. If the member has reached their target, he or she will receive a reward from the entity or its representative. The computer system generates an aggregate report for the entity that illustrates the changes in physiological condition for the group of members of the entity and this report is reviewed in process 107.

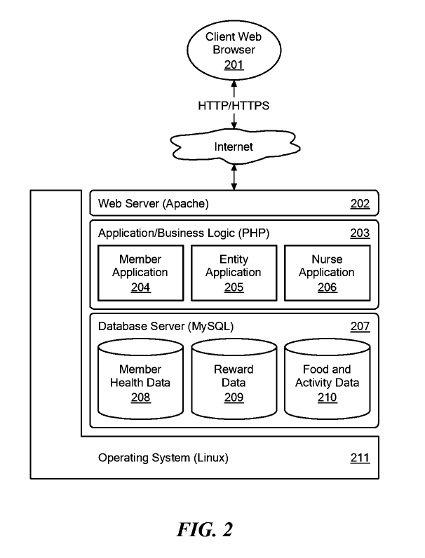

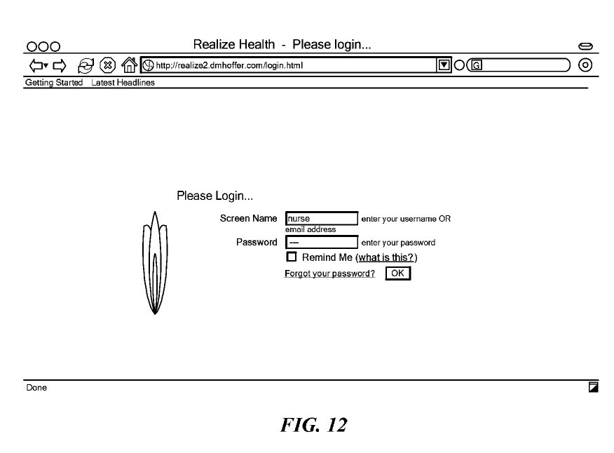

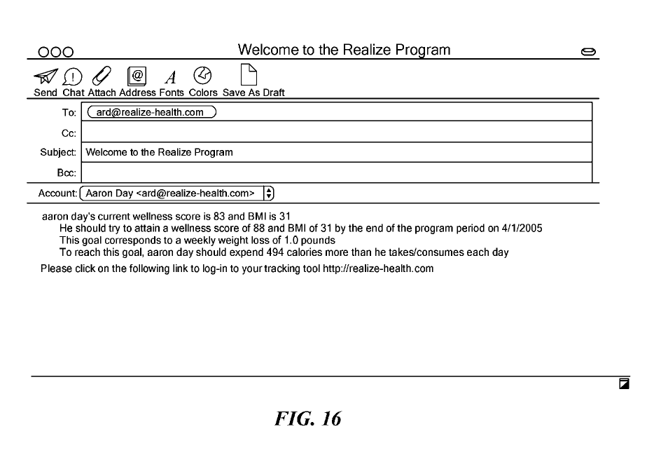

FIG. 2 is a block diagram illustrating a software architecture for the system of FIG. 1. In one of many embodiments of the system, an application is developed using a three-tiered open-source platform. Such an architecture may include a web browser 201 through which a member may access the system. Such a web browser may include, but is not limited, to Internet Explorer, Netscape, Safari, Firefox, AOL, and/or Mozilla. The application may be served to the member using an Apache (or other) web server 202. In accordance with embodiment of FIG. 2, an application/business logic layer 203 may be developed using the PHP programming language. One such PHP application may be a member application 204 for tracking and incentives. Such a feature is shown in FIGS. 16, 17, 18, and 19. One of the PHP applications may also be an entity application 205 for administering the program. Another of the PHP applications may be a nurse application 206 for collecting the member's physiological condition information as shown in FIGS. 12, 13, 14, and 15.

The architecture may include a database server 207 and the database layer may be developed using MySQL. Member health data 208 may be stored in a MySQL database created for storing the member health data. A MySQL database may also be created for storing reward data 209 related to the members and groups of members. Similarly, a MySQL database may be used to store caloric and nutritional information 210 about food items and caloric information about exercise activities. The system may be run on a Linux or other operating system 211. Note that data may be collected through a website or other wired method. Similarly, data may be collected via a wireless device such as a PDA or cell phone. For example, physiological condition measurements may be generated and sent via small, wearable devices, e.g., physiological sensor, pedometers, GPS devices such as a GPS watch, etc., that may network with one another or with a computer system.

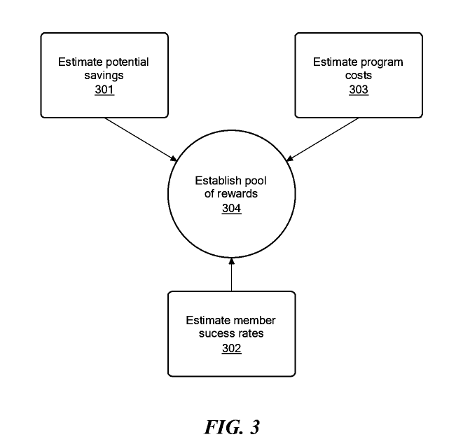

FIG. 3 is a flow chart illustrating a system for publicizing a pool of rewards. The pool of rewards may be established based on a sharing of the savings determined by examining how the particular variables interrelate. In process 301, potential savings are estimated. In one embodiment, such savings are estimated using available tools related to health care cost savings such as the Obesity Cost Calculator provided by the National Business Group on Health, Washington D.C., by which an entity can estimate the cost savings/productivity increase associated with improvement in physiological condition. Table 1 represents sample data from the Obesity Cost calculator that might be used to estimate potential savings for an entity. Obesity, in this example, is defined as having a BMI (Body Mass Index) in excess of 30. BMI is computed by taking [weight in lbs/(height in inches)²]*703.

Using the Obesity Cost Calculator, one may similarly determine costs, for example, for an employee who is not obese but is overweight. As discussed in further detail below, determinations such as these can be used for associating a presumed cost savings for an individual who, over some period of time, improves health status, for example, from obese to merely overweight or from overweight to healthy.

TABLE 1

| Inputs | Industry | Manufacturing |
|---|---|---|
| | State | Massachusetts |
| | # Employees (and spouses) | 6000 |
| | Avg. Employee Hour | $25.33 |
| | compensation w/o Benefits | |
| | Avg. Employee Hour | $33.68 |
| | compensation w/benefits | |
| | % of Employees w/benefits | 100% |
| Outputs | Medical Costs | $1,932,074 |
| | Productivity Costs | $402,112 |
| | Total Costs | $2,334,186 |
| | Total Costs/employee | $389 |
| | Total Costs/obese employee | $1,145 |

Table 2 is a table showing physiological conditions according to BMI.

TABLE 2

| BMI Range | Physiological Condition |
|---|---|
| >30.0 | Obese |
| 25-30 | Overweight |
| 18.5-25 | Normal |
| <18.5 | Underweight |

Member success rates are estimated in process 302. In one embodiment, the member success rates are based on concrete measurements in physiological condition that directly relate to either a cost savings or an increase in employee productivity. Member success rates are based on factors such as percentage of participation, percentage of successes, percentage of successes for two or more consecutive time intervals. Table 3 demonstrates possible changes in physiological condition as well as potential savings/member for each change in physiological condition.

In one embodiment of the invention, the average quarterly savings is computed by taking the total costs for an obese employee as determined in Table 1 and dividing that number by 4. A similar process is repeated for an overweight employee using the Obesity cost calculator. Table 3 was determined using 1145/4=286.25/quarter costs for an obese person and an assumed $100/quarter in additional costs for an overweight employee. The average quarterly cost savings for an employee that was initially obese and end the quarter overweight was computed by subtracting the quarterly overweight costs from the quarterly obese costs ($286.25−$100=$186.25). In this embodiment, it was assumed that a healthy person adds $0 in additional costs. The average quarterly savings for a member that was initially obese and ended the quarter healthy was computed by subtracting the quarterly additional healthy costs from the quarterly obese costs ($286.25−$0=$286.25). Finally, the average quarterly savings for a member that was initially overweight and ended the quarter healthy was computed by subtracting the quarterly additional healthy costs from the quarterly overweight costs ($100−0=$100)

TABLE 3

| Initial Physiological | Final Physiological | |
|---|---|---|
| Condition | Condition | Average Quarterly Savings |
| Obese | Overweight | $186.25 |
| Obese | Healthy | $286.25 |
| Overweight | Healthy | $100 |

In process 303, program costs are estimated. In one embodiment, the program costs to an entity may include but are not limited to marketing and communications expenses, project management resources, tracking tool development and hosting costs, measurement costs, and computer hardware and software costs. In process 304, a pool of rewards is established. In one embodiment, the entity will factor all of the potential costs and savings from above and will then test different reward levels for both members and groups of members to determine the overall impact on profitability. Members may be successful in improving their physiological condition over the course of the time period; however, such success might not result in a change in savings for the entity. For instance, a member may successfully lose 12 lbs. during a quarter. Although they were successful in reaching their individual goal for the period, their initial and final physiological condition may still be obese. Table 4 demonstrates several reward pools that may be created.

TABLE 4

| Initial Physiological | Final Physiological | |
|---|---|---|
| Condition | Condition | Reward |
| Obese | Obese | $10 |
| Obese | Overweight | $25 |
| Obese | Healthy | $100 |
| Overweight | Overweight | $10 |
| Overweight | Healthy | $25 |
| Healthy | Healthy | $10 |

Groups of members and entities may also compete for prizes based on greatest average improvement of the members of a group or entity. Table 5 represents a possible reward pool for groups and entities:

TABLE 5

| Ranking | Reward |
|---|---|
| 1^{st} Place | $5000 |
| 2^{nd} Place | $2500 |
| 3^{rd} Place | $1000 |

According to one embodiment of the invention, the overall rewards may be determined as a percentage of the savings. In one embodiment, the rewards may be determined by factoring the assumed reward values (such as shown in Table 4) with presumed percentage success rates (based on initial and final physiological conditions as listed in Table 3) for members in the program, presumed program costs and overall savings to the entity.

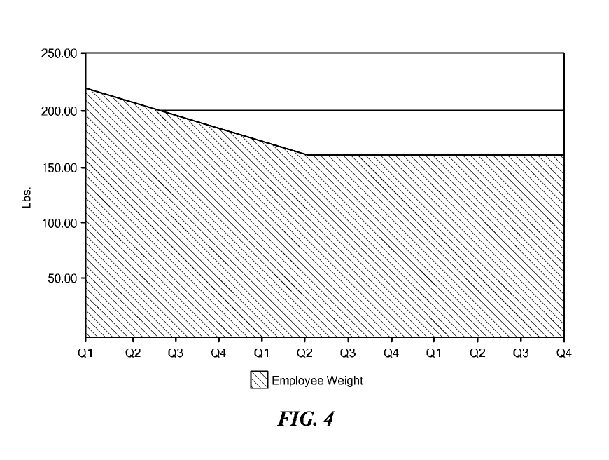

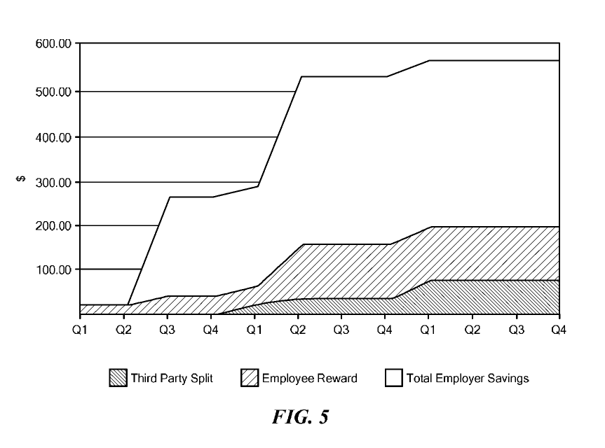

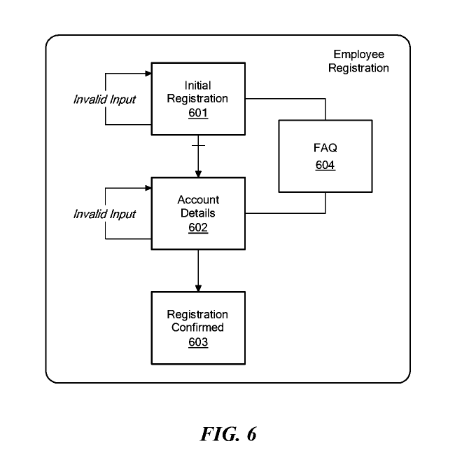

FIG. 4 is a graphical illustration showing the change in weight for a member over a 3 year time period for a member who was initially 5′9″ 220 lbs. FIG. 5 is a graphical illustration showing how the distribution in savings from the system based on a certain pool of rewards and distribution to a third party for managing the system relates to the weight loss demonstrated in FIG. 4. The total savings was determined by computing the member cost for the quarter based on the physiological condition at the end of the quarter from initial cost based on the member's initial physiological condition. The employee reward is based on the table of individual rewards above. FIG. 6 is a flow chart illustrating the process for a member enrolling in the system.

A member initially registers online in process 601. During the registration process, the member may access, in process 602, an interface that provides the member with answers to frequently asked questions related to the system. The member inputs account details such as specific member information in process 603. The member receives, in process 604, a confirmation of the registration.

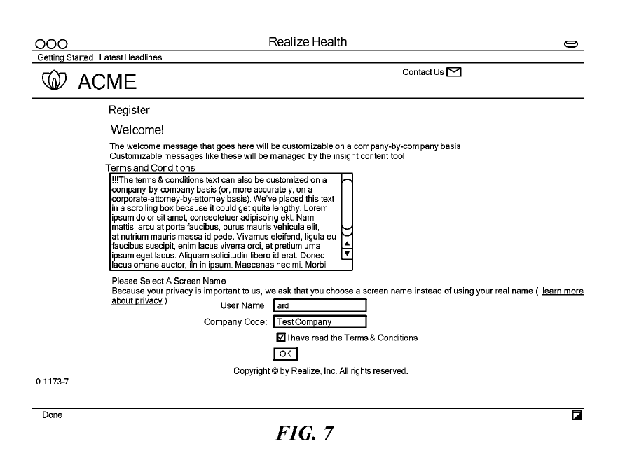

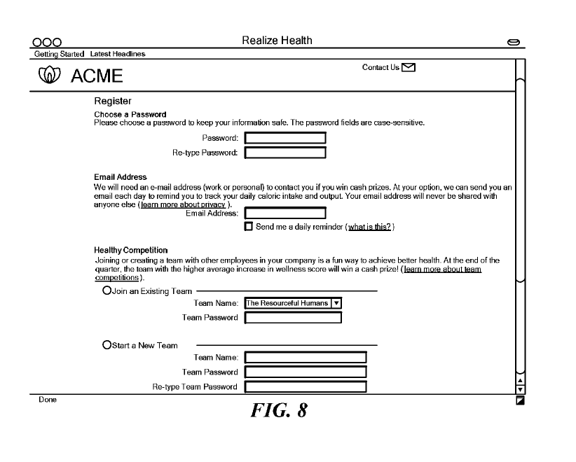

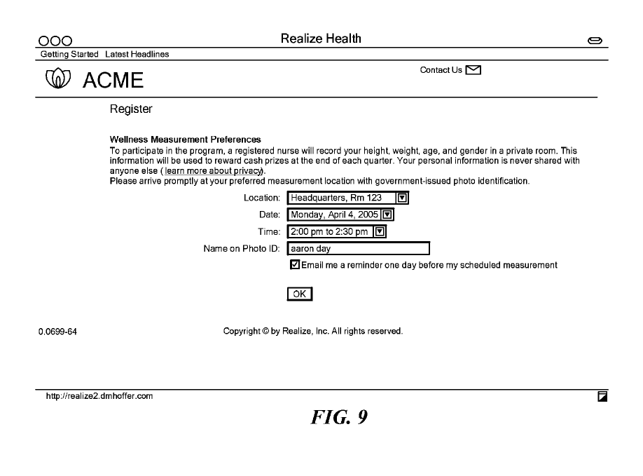

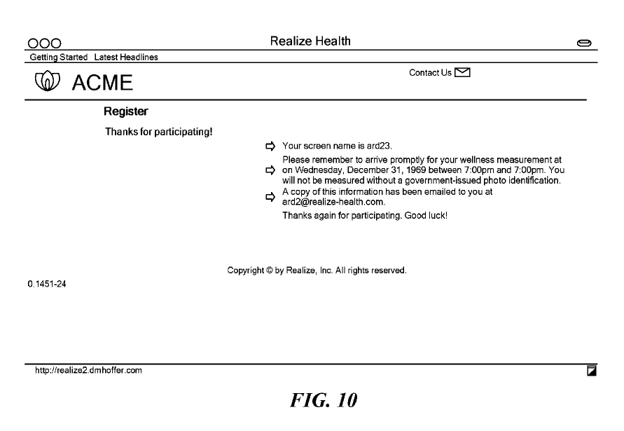

FIG. 7 is a screen shot design for a log-in page for a website tracking tool for a member. In one embodiment, a member can sign up selecting a username and company code. This embodiment allows for privacy and is fully HIPAA compliant. FIG. 8 is a screen shot design for a member enrolling in the program through a website. In one embodiment, the member is then able to select a personal password for their account and, at their options, join other members as a part of a team. FIG. 9 is a replica of screen shot design for a member providing authentication as to their individual identity. In one embodiment, once the member has filled out the registration information, the member is asked to provide their name as indicated on a Photo ID. The member's identification will be validated by the entity or a third party in order to maintain the integrity of the program and to ensure that the reward pool is distributed accurately. FIG. 10 is a screen shot design for a registration confirmation page for a member of an entity. In one embodiment, the member is provided with a confirmation screen that contains a date, time, and location for official measurement.

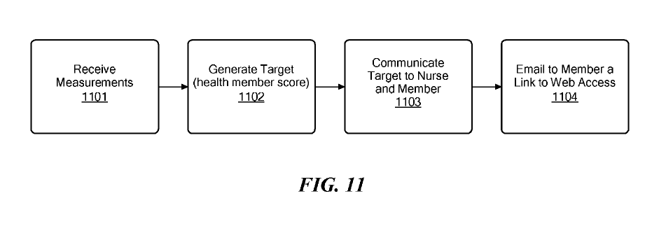

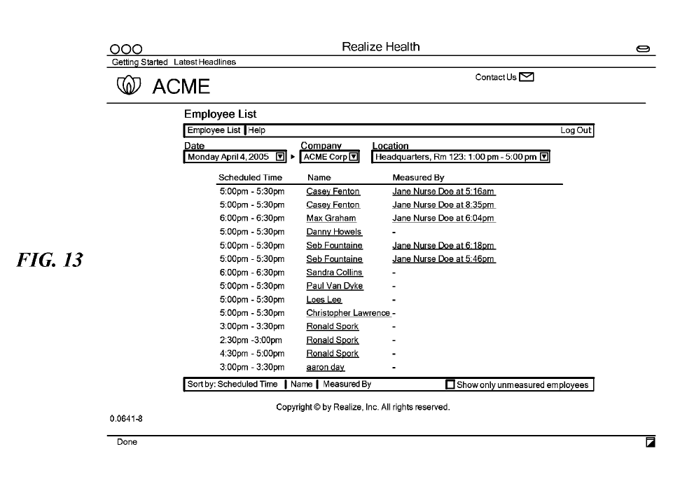

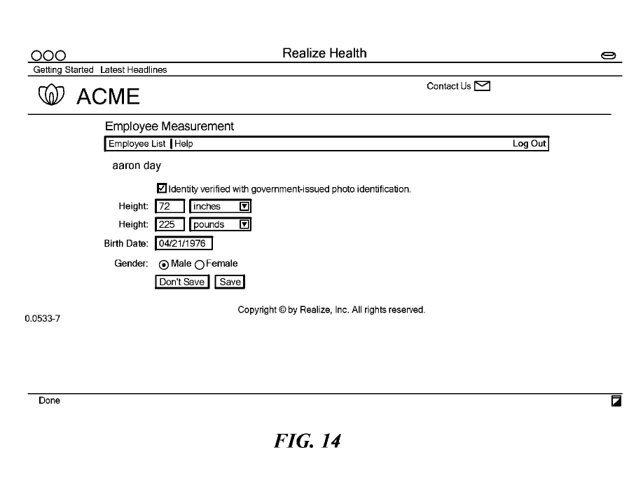

FIG. 11 is a flow chart illustrating the process of measuring a member's physiological condition. The system receives measurements related to a member's physiological condition in process 1101. In accordance with one embodiment, a website allows the entity or its representative to log height, weight, gender, and date of birth. FIG. 12 is a screen shot design for a website log-in page for an application that records members' physiological condition and stores them in a database. FIG. 13 is a screen shot design for a website scheduling page that allows an entity to coordinate the logistics of measuring the physiological condition of members of an entity. FIG. 14 is a screen shot design for a website page used for recording a specific member's physiological condition;

A target/health member score is generated in process 1102. In one embodiment, the system's database contains an algorithm that is used to compute a target physiological condition for each member. The algorithm is developed based on the ability of the member to achieve an improved physiological condition based on healthy guidelines as established by reputable sources. In one embodiment, the algorithm used for calculating the physiological condition is percent based on BMI (which is derived from height and weight measurements) and percent based on blood pressure (based on systolic and diastolic measurements). Other variables include smoking behavior, cholesterol levels, blood sugar level and EKG, for example, which may be incorporated in the physiological condition score. For example, the physiological condition may be determined by 50% BMI, 20% blood pressure, 20% smoking behavior, and 10% cholesterol. Although new variables may be added to the score and algorithmically changed their relative weightings in computing the physiological condition, the index does not change. In other words, regardless of how the number of variables and complexity of the algorithm changes, the concept of an overall wellness score based on an index representative of the physiological condition does not change. In specific embodiments, the score may be determined within an index from 0 to 100.

In summary, the physiological condition is a way to measure a number of variables related to human health, apply an algorithm to those variables, and provide the individual with a physiological condition based on their gender and age range that will let them know their relative health. An individual will know whether they have a wellness score of 50 or 90 and can begin modifying their behavior to attempt to attain a better wellness score or maintain an existing wellness score consistent with wellness. In one embodiment, the algorithm for the wellness score is based on the initial BMI for the member and the distance away from the target healthy BMI for that member. According to this embodiment, if the member is overweight or obese, the wellness score is calculated by the following formula (100−3×(current BMI−target BMI (25)). If a member has a BMI of 33, for instance, their wellness score would be (100−(3×(33−25))=76. A BMI between 18.5-25 corresponds to a wellness score of 100. If a member is underweight (has a BMI<18.5), then the wellness score is computed by the following formula (100−3×(18.5−Current BMI). So, if a member has a BMI of 15, their wellness score would be (100−3(18.5−15))=89.5. If the member is overweight or obese the target physiological condition for that quarter is the lesser of the target wellness score computed by subtracting 12 lbs. from current weight or the difference between the current weight and the weight for the member corresponding to a BMI of 24.9. If the member is underweight the target physiological condition for that quarter is the lesser of the target wellness score computed by adding 12 lbs to the current weight or the difference between the weight for the member corresponding to a BMI of 18.5 minus the current weight.

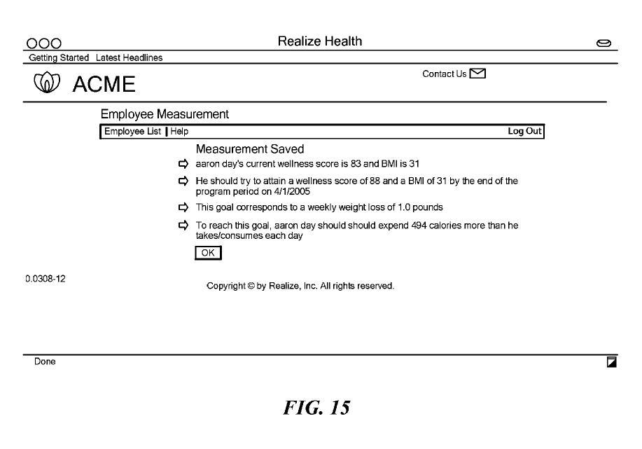

Target information is communicated to the “nurse” and to the member in process 1103. In one embodiment, after the measurement information has been recorded into the system by the entity or a third party, the system provides a website confirmation of the current physiological condition of the member and sets a target for the member. FIG. 15 is a screen shot design for a website page that confirms the member's current physiological condition and provides a physiological target score for a specified time period.

A link to web access is emailed to the member in process 1104. In accordance with one embodiment the entity or a third party will e-mail the member a link to a website containing the members current and target physiological condition. FIG. 16 is a screen shot design for an e-mail confirmation for a member that has signed up for the program.

The invention may include a tracking tool that sets an individual wellness target over a specific period of time and allows the individual to track certain behaviors that influence the variables that comprise the physiological condition. In one embodiment, one variable is tracked in the wellness index (BMI). The best way to lower body mass is to burn more calories than are consumed. Accordingly, in one embodiment of the invention, the focus will be on caloric intake and caloric expenditure (burn). Other embodiments of the invention include tracking other variables such as smoking behavior, alcohol consumption, etc.

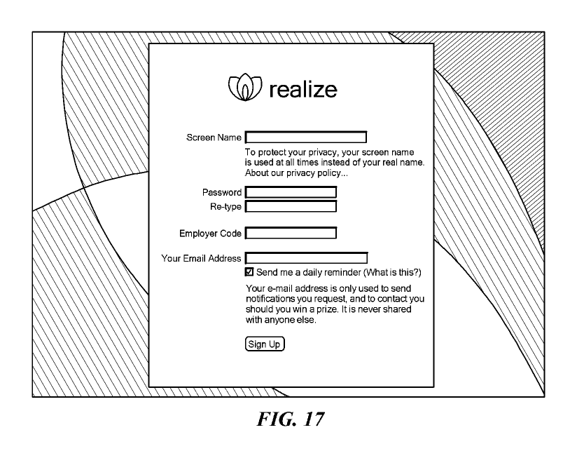

FIG. 17 is a screen shot design for a log-on page for a tracking tool that allows members to monitor their physiological condition. This is the log-on screen for the individual user of the tracking tool according to one embodiment of the invention. Each individual may have a screen name. The member's actual name may not be used so as to protect the privacy of the member's health information.

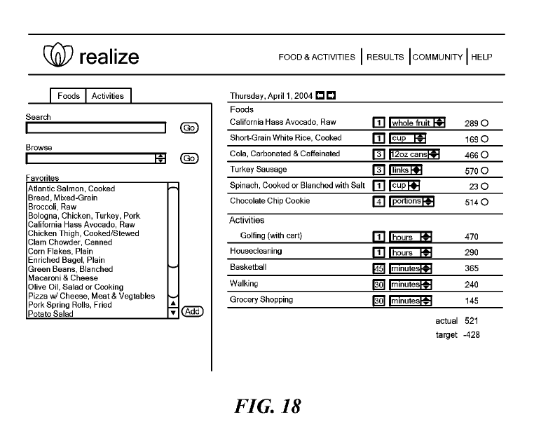

FIG. 18 is a screen shot design for a website page that allows a member to track their caloric intake and caloric expenditure against their individual goal. In accordance with one embodiment, the member tracks the caloric intake/expenditure. This is the area of the tracking tool where the member tracks the type or quantity of food they have consumed during the course of the day. Based on the data gathered in calculating the physiological condition score, each individual's basal metabolism will be known (the amount of calories burned without any daily activity). Based on the target wellness score a daily target number of calories to be burned based upon the basal metabolism and activity level is provided. This section of the tool tracks total caloric intake and caloric breakdown based upon healthy eating guidelines for energy, nutrients, and vitamins. In one embodiment of the system, the guidelines are derived from the USDA or from the Healthy Eating Pyramid and may be modified over time as more information becomes available concerning the optimal balance that should be achieved in a human diet. This is also the portion of the system's tracking tool where the individual logs the number of calories burned based on activity type and duration. The tracking tool automatically takes into consideration the individual's body height and weight in determining the number of calories burned for each activity/duration level.

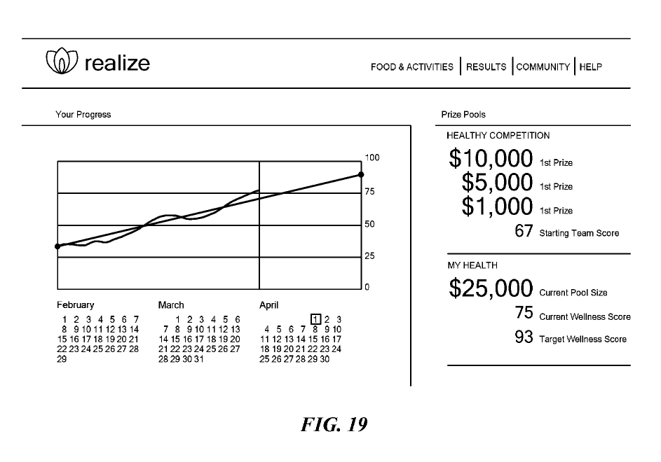

FIG. 19 is a screen shot design for a website page that allows members to monitor their progress against their goal. The graph shown in FIG. 19 ties in all of the previous elements including caloric intake, caloric burn, physiological condition, and adds a new reward component. On this graph members can see what their physiological condition was at the beginning of a given time, their physiological conditional target for the end of the period, and, based on the caloric intake, caloric burn information the individual has entered into the system, the individual's success in meeting their own goals can be observed. The calendar shows the days in which the individual met or exceeded his or her goal (green), days in which the individual did not reach his or her goal (red), and days in which the data the individual entered was incomplete (gray). The member can go back and modify the data from any day in the past. This screen also shows the total reward prize pool (to be explained in the next section) and displays the total number of other participants in the program.

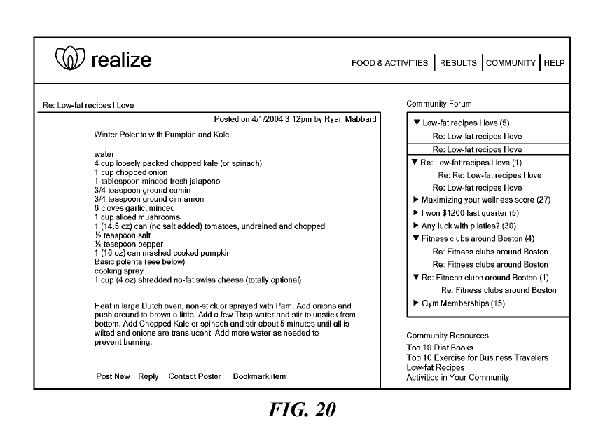

FIG. 20 is a screen shot design for a website page that allows a member to communicate and interact with other participants in the system. In one embodiment, members can share recipes, diet and exercise tips, and links to various resources that may be helpful to them.

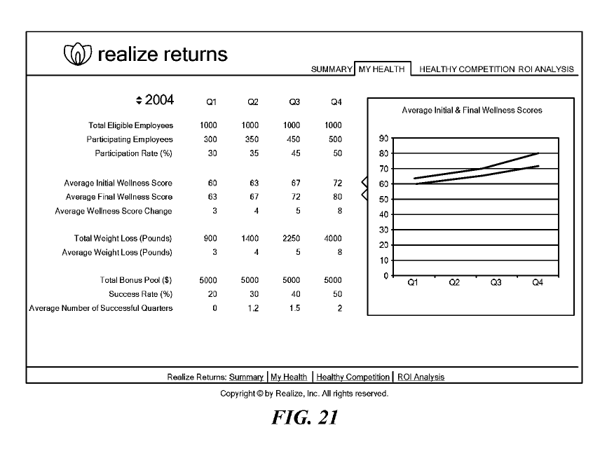

FIG. 21 is a screen shot design for a website page that allows an entity to view the aggregate success of their members. In one embodiment of the invention, the entity can view the aggregate number of eligible participants, participation rate, improvement in wellness score, total lbs. lost, total size of the bonus pool, etc.

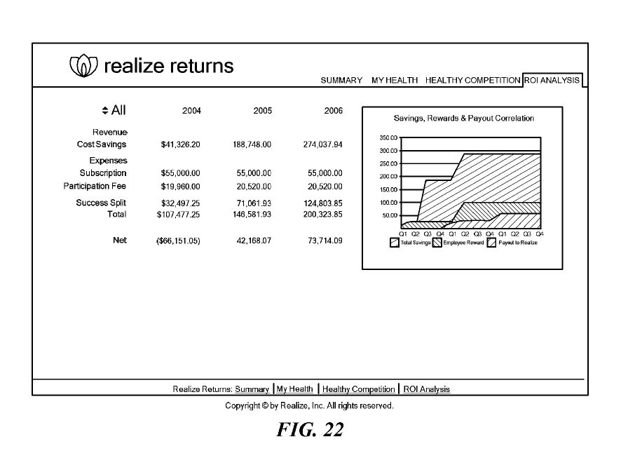

FIG. 22 is a screen shot design for a website page that allows an entity to view their financial return for the program factoring in program costs and the distribution of the pool of rewards. In one embodiment of the invention, the entity can view the total amount of savings, distribution of total savings to the entity, member, and a third party program administrator. In one embodiment this webpage also shows the total savings and total costs of the program to the entity including marketing, communication, and measurements cots.

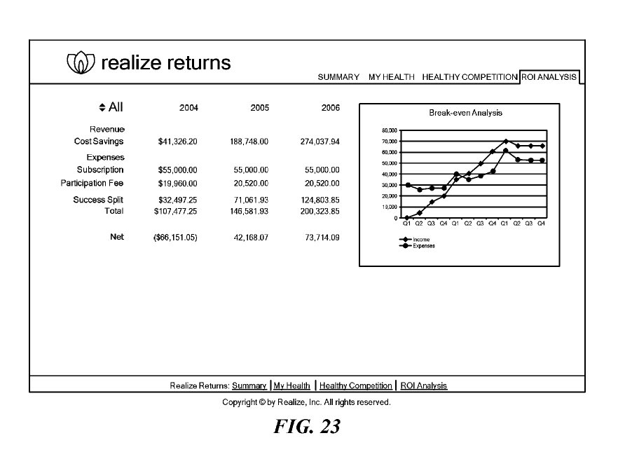

FIG. 23 is a screen shot design for a web page that allows an entity to view their overall breakeven analysis for the program period. In one embodiment of the invention, a breakeven point is determined using cost savings for revenue and program costs for expenses.

Note: A third party can do all of the following on behalf of an entity.

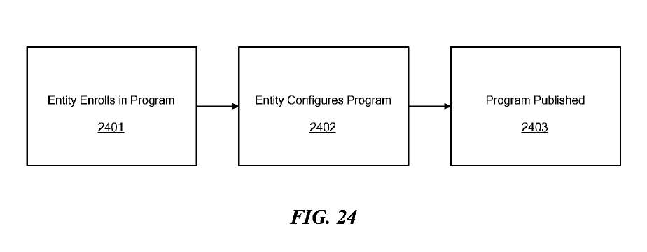

FIG. 24 is a block diagram illustrating logical flow for entity enrollment in a program in accordance with an embodiment of the present invention. The entity may be a corporation, a governmental body, a non-profit organization, a family, a university, a public or a private school that enrolls in the program through a series of web pages.

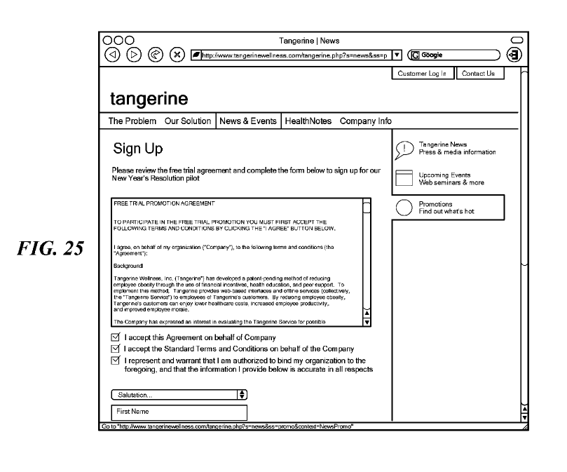

FIG. 25 is a representation of a web page presented to a representative of an entity for enrolling in the program of FIG. 24. In this embodiment, the enrollment web page allows the entity to enroll using a click-wrap web page contract.

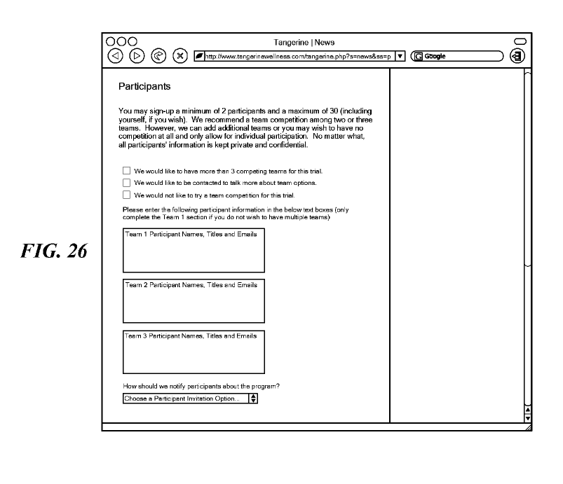

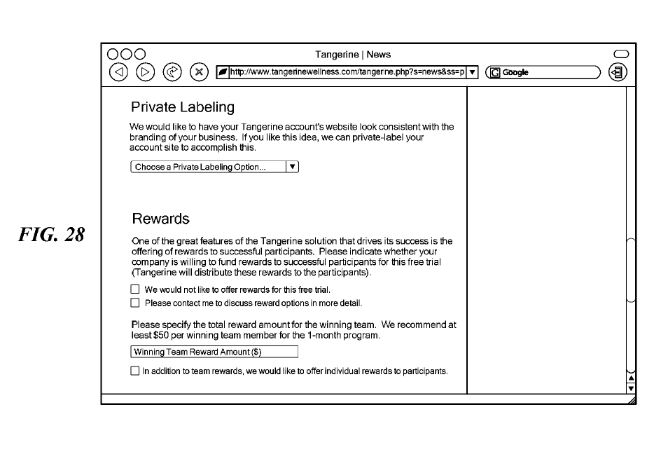

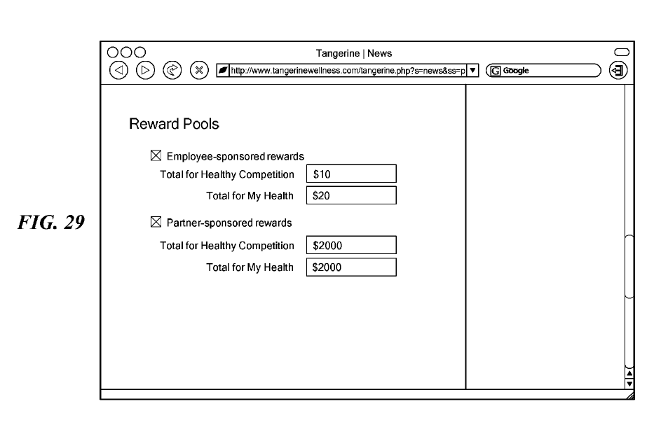

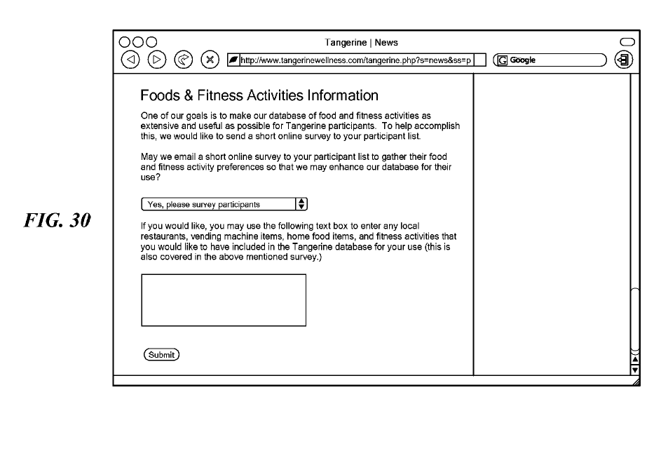

FIGS. 26, 28, 29 and 30 are representations of web pages presented to a representative of an entity for configuring the program of FIG. 24. In this embodiment, using a web page, the entity configures the type and amount of rewards for both individuals or teams, sources of reward funds, team composition (names, e-mail address, titles, location), entity website branding, and food and activity information that is pertinent to the entity's users.

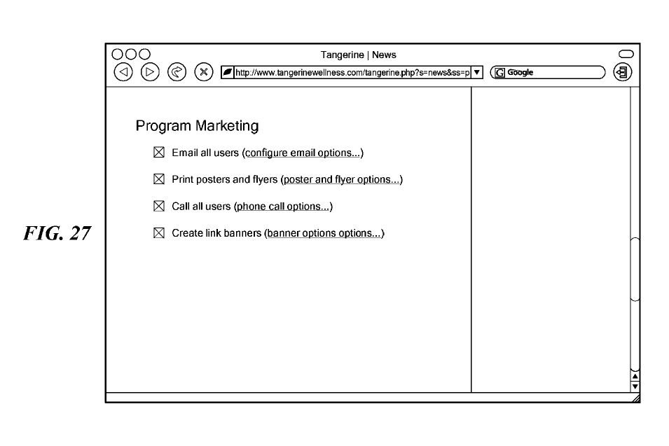

FIG. 27 is a representation of a web page presented to a representative of an entity for handling publication of the program of FIG. 24. In this embodiment, through a webpage, an entity can select one or more of the following types of program marketing options: e-mail, printed posters and flyers, telephone calls, web banners and links.

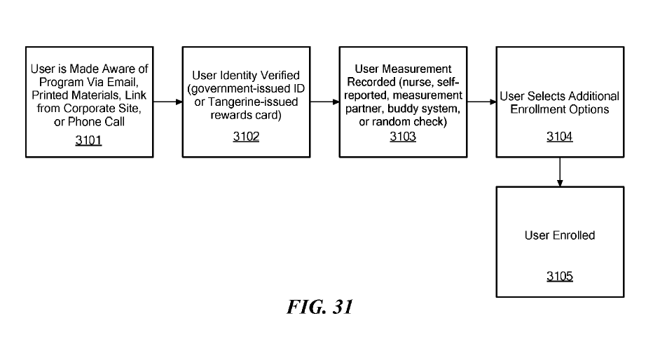

FIG. 31 is a block diagram, in accordance with an embodiment of the present invention, illustrating logical flow associated with user enrollment in the program of FIG. 24. In this embodiment, a user enrolls online through a set of steps that may involve a third party verifying user identification.

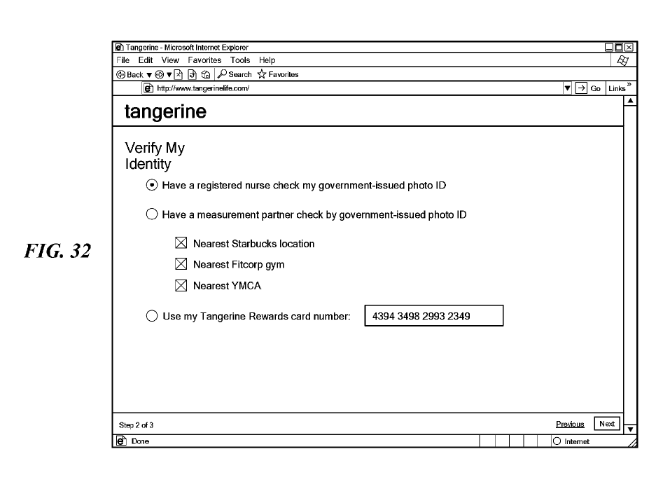

FIG. 32 is a representation of a web page, pursuant to the logical flow of FIG. 31, presented to a user enrolling in the program of FIG. 24 for handling verification of identification. In this embodiment, the user's identification is verified by a nurse, by a measurement partner, or using a rewards card.

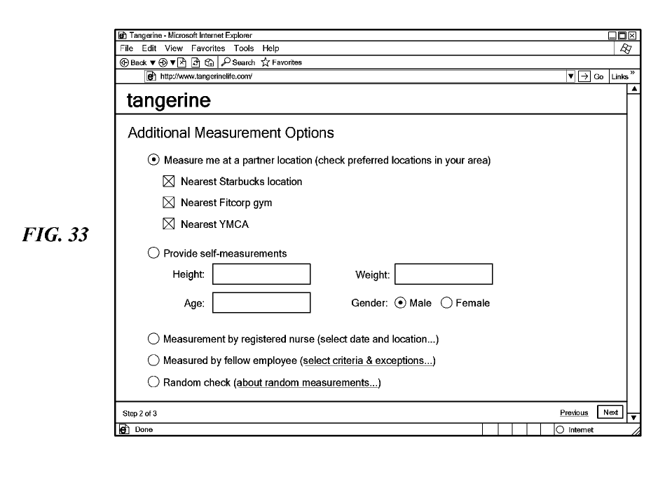

FIG. 33 is a representation of a web page, in accordance with an embodiment of the present invention, presented to a user enrolling in the program of FIG. 24 for handling weight measurement options. In this embodiment, the user may select partner locations where the user's measurements may be recorded. Alternatively, the use may choose to enter measurements made by the user.

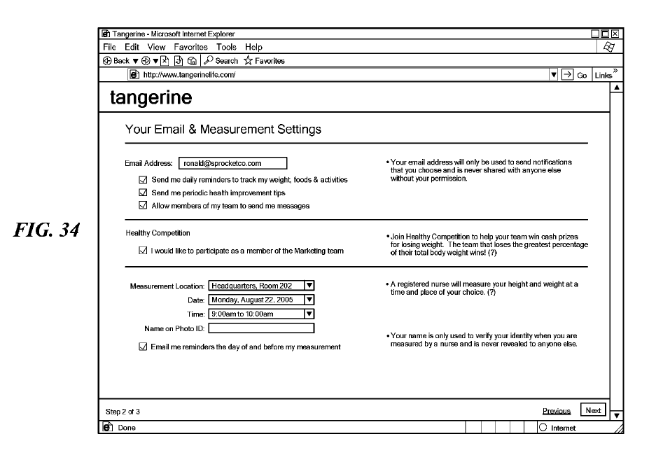

FIG. 34 is a representation of a web page, in accordance with an embodiment of the present invention, presented to a user enrolling in the program of FIG. 24 for handling communication and election of team participation. In one embodiment, using a web page, the user can modify their e-mail address, elect to participate on a team, specify measurement location, and choose to receive measurement reminder e-mails.

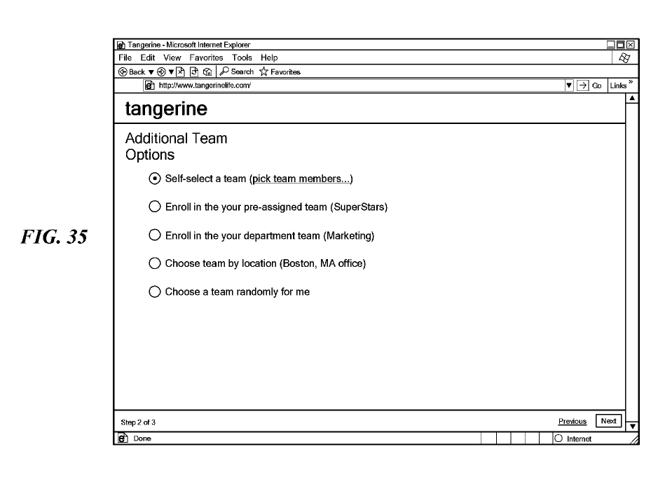

FIG. 35 is a representation of a web page, in accordance with an embodiment of the present invention, presented to a user enrolling in the program of FIG. 24 for handling team participation options. In one embodiment, using a web page, the user can select a team on which to participate. In one embodiment, teams can be user-selected, pre-assigned, department based, location based, or assigned randomly.

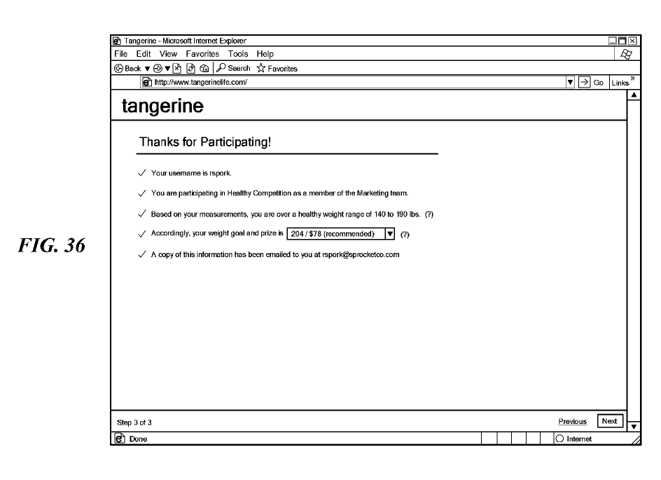

FIG. 36 is a representation of a web page, in accordance with an embodiment of the present invention, presented to a user confirming the user's enrollment in the program of FIG. 24. In one embodiment, a confirmation webpage displays the username, team designation, current weight classification (over or under a healthy weight), proposed target weight, and e-mail address of the user. In one embodiment, the user may select their own measurement goal (within a healthy range).

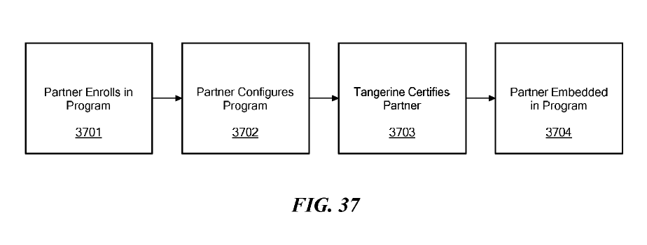

FIG. 37 is block diagram of logical flow, in accordance with an embodiment of the present invention, associated with enrollment of a partner in the program of FIG. 24. In one embodiment, using a series of webpages, a partner may enroll, configure the program, obtain web-training, obtain certification, and become embedded in the program.

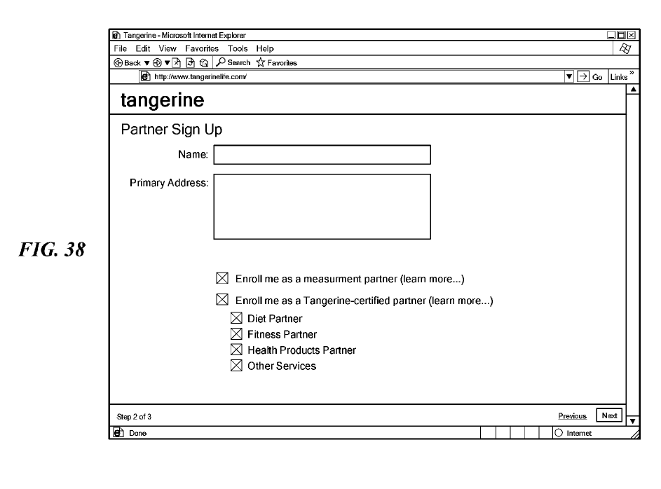

FIG. 38 is a representation of a web page, in accordance with an embodiment of the present invention, presented to an organization representative for enrollment of the organization as a partner in the program of FIG. 24. In one embodiment, using a web page, the user enters name, address, and partner type.

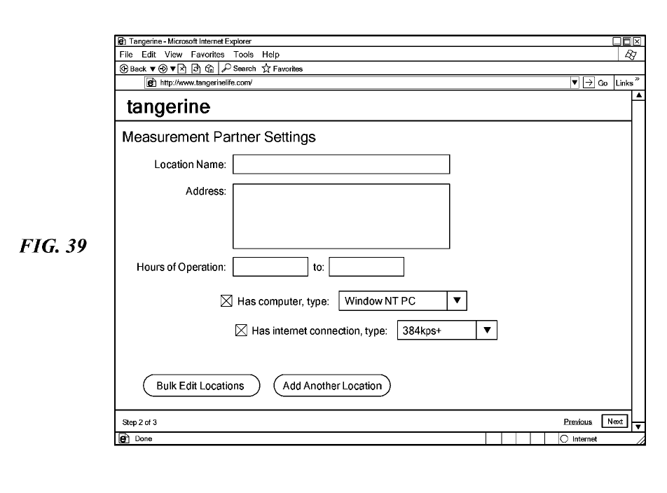

FIG. 39 is a representation of a web page, in accordance with an embodiment of the present invention, presented to the organization representative for providing data pertinent to operating as a measurement partner in the program of FIG. 24. In one embodiment, using a web page, the partner specifies location name, address, operating hours, computer type, and internet connection speed for one or more locations.

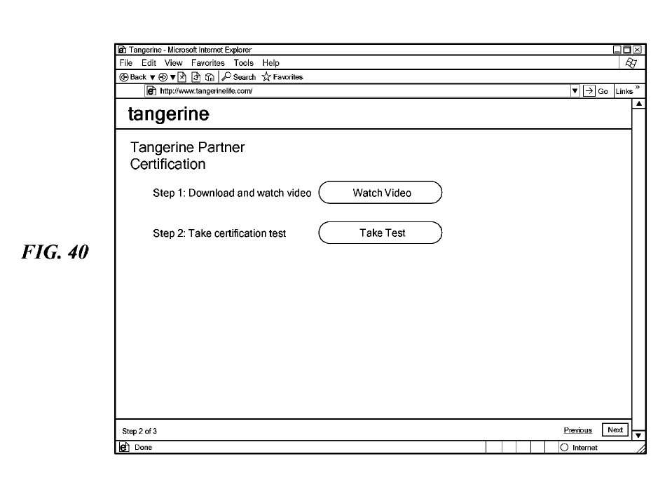

FIG. 40 is a representation of a web page, in accordance with an embodiment of the present invention, presented to the organization representative for training and certification pertinent to operating as a measurement partner in the program of FIG. 24. In one embodiment, through a web page, the user watches training videos and takes certification tests.

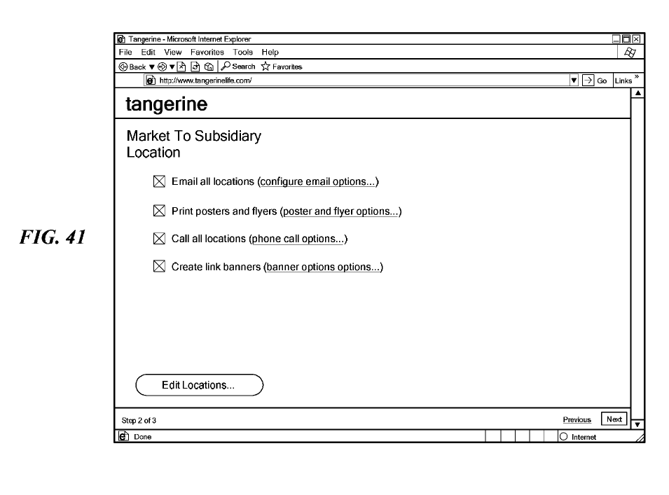

FIG. 41 is a representation of a web page, in accordance with an embodiment of the present invention, presented to the organization representative for publicizing to subsidiary locations of the organization the fact that the organization has become a partner in the program of FIG. 24. In one embodiment, using a web page, the partner can select and configure various marketing options to subsidiary locations (e-mail, printed posters and flyers, telephone calls, and web banners and links).

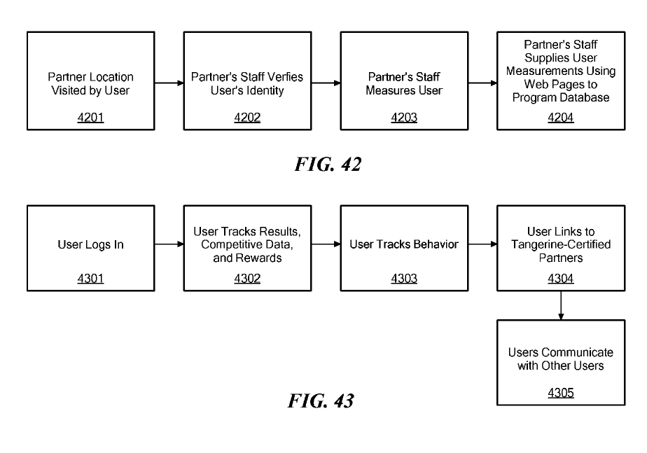

FIG. 42 is a block diagram showing a process, in accordance with an embodiment of the present invention, by which staff of a measurement partner obtains measurements of a user and provides them using web pages over a network to a database. In this embodiment, after a partner facility is visited by a user in process 4201, a Partner's staff member verifies the identity of the user in process 4202, ascertains measurements of the user in process 4203, and, in process 4204, uses one or more web pages to provide the user measurements to the database over a network. Typically the Partner will have internet access, and the web pages provided by a server over the internet, and the server will be in communication with the database.

FIG. 43 is a block diagram, in accordance with an embodiment of the present invention, showing processes by which a user obtains information concerning behavior and results of participation in the program of FIG. 24 and communicates with partners and others. In one embodiment, using one or more web pages, the user logs in, tracks results, and obtains information concerning competitive data, rewards, and behavior, has links to partners, and can communicate with other users.

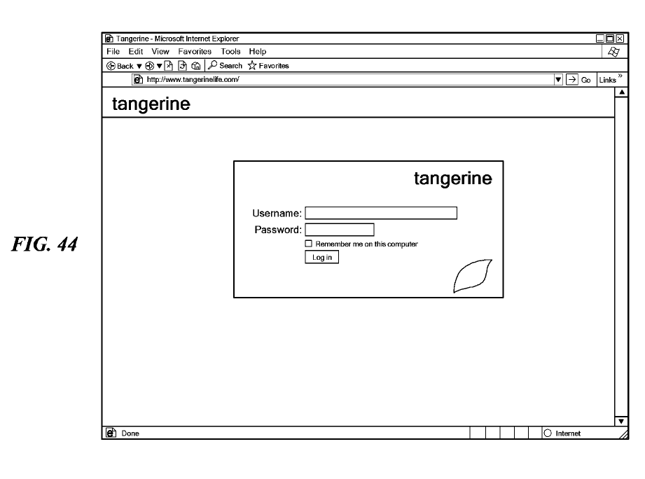

FIG. 44 is a representation of a web page, in accordance with an embodiment of the present invention, presented to the user for logging in to obtain the information of FIG. 43. In this embodiment, using a web page, the user enters a username and password. In a related embodiment, the user's username and password information may be stored locally on the user's computer to speed the log-in process.

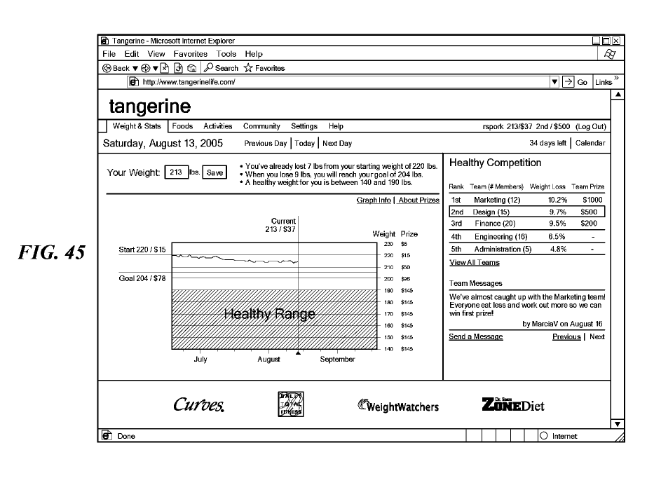

FIG. 45 is a representation of a web page, in accordance with an embodiment of the present invention, presented to the user for tracking results of participation in the program, viewing competitive data, communicating with teammates, and receiving advertising content. In this embodiment, using a web page, the user records the user's weight, views the user's progress towards an individual weight goal, views team rankings, sends messages to teammates, and views advertisements.

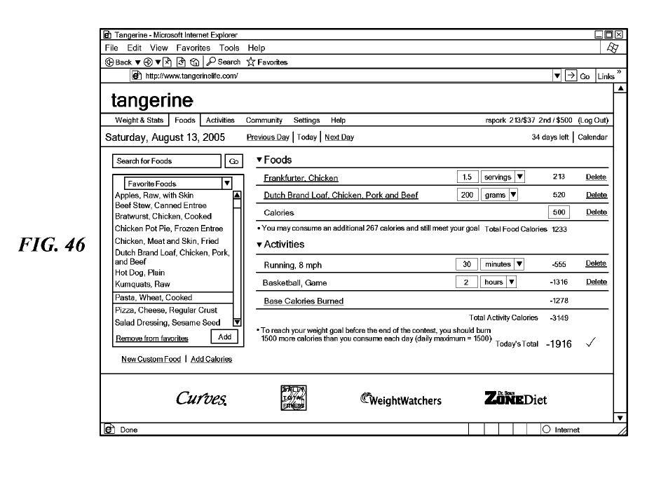

FIG. 46 is a representation of a web page, in accordance with an embodiment of the present invention, presented to the user for tracking behavior and receiving advertising content. In this embodiment, using a web page, the user logs calories consumed, calories burned, logs custom foods and activities, creates a favorite foods and activities log, views progress towards daily caloric balance goals, and views advertisements.

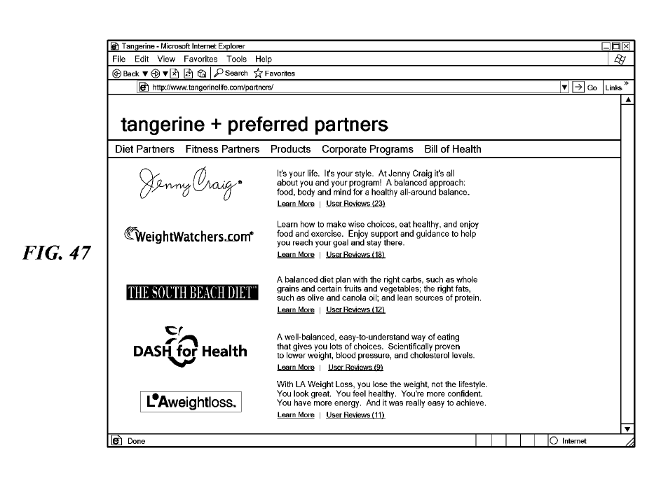

FIG. 47 is a representation of a web page, in accordance with an embodiment of the present invention, presented to the user for viewing offerings by partners enrolled in the program of FIG. 24. In this embodiment, using a web page, the user views offerings from diet, exercise, restaurant, health content, exercise equipment, vitamin and supplement, and other partners that have offerings related to program success. In a further related embodiment, the users can provide feedback and ratings about the partners. In that embodiment, this user feedback can be used optionally to make recommendations to other users participating in the program.

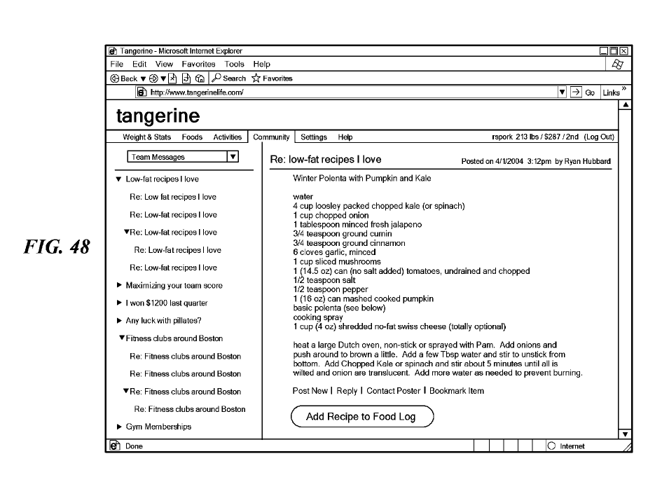

FIG. 48 is a representation of a web page, in accordance with an embodiment of the present invention, presented to the user for sharing messages (for example recipes) with other users. In this embodiment, using a web page, the user can post supportive messages, recipes, diet and exercise resources and tips, and other information to other users.

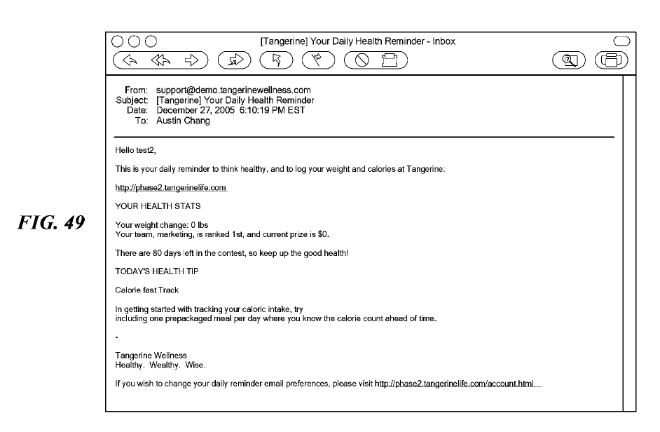

FIG. 49 is a representation of a supportive e-mail message, in accordance with an embodiment of the present invention, to the user providing competitive and behavior data. In this embodiment, the user receives a program generated e-mail that contains a daily reminder, current health statistics, reward information, and health tips.

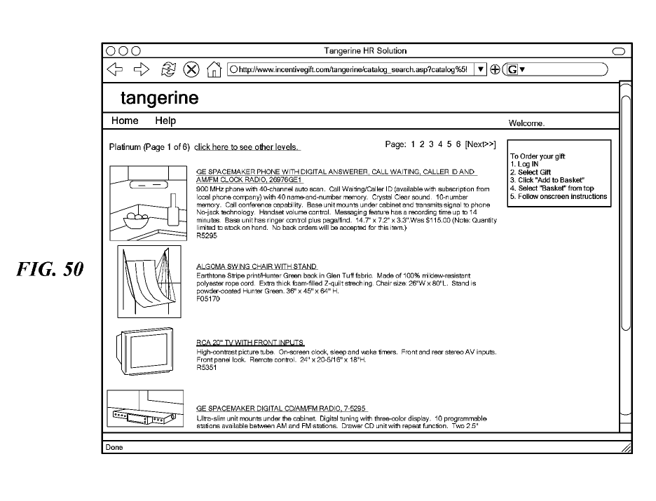

FIG. 50 is a representation of a merchandise web page, in accordance with an embodiment of the present invention, presented to the user for displaying merchandise that may be obtained using reward points obtained by participation in the program. In this embodiment, using one or more web pages, the user can redeem merchandise, vacations, gift cards, and other gifts using points earned through the program.

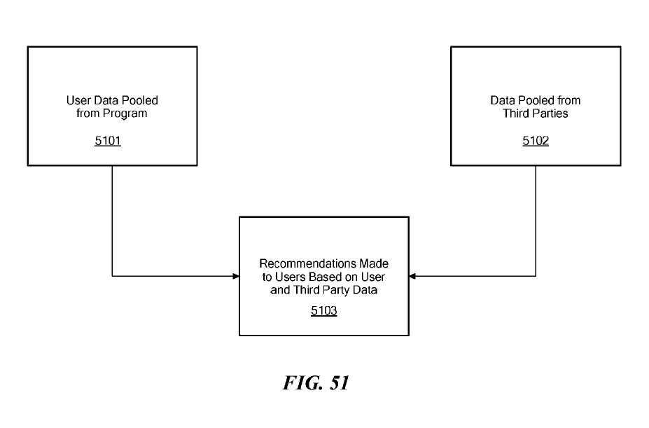

FIG. 51 is a block diagram showing how recommendations to users in the program are based on both pooled user data and on third party data. In this embodiment, user data, third party data, and other user's data, generates recommendations that are displayed on a web page. In related embodiments, the aggregate data is used for targeting partner advertisements. Embodiments may use the actual success of program participants to determine the cost or charge to the partners to be included or listed as a resource. For example, a partner that is highly successful among the users may be charged less than a partner that is less successful or vice versa.

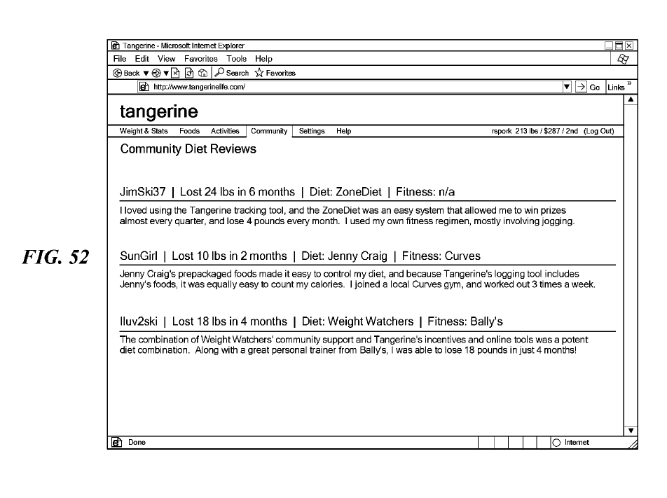

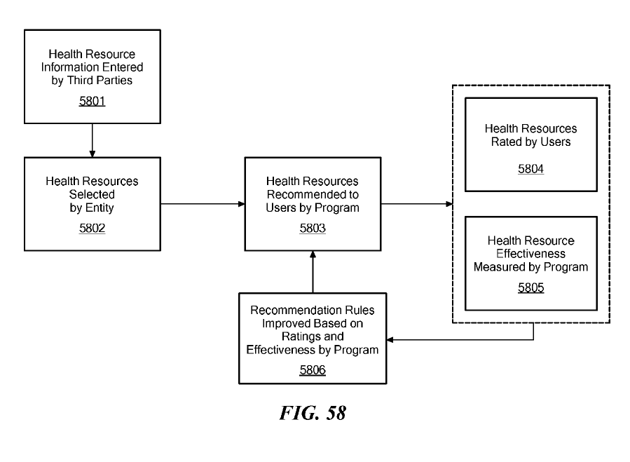

FIG. 52 is a representation of a web page, in accordance with an embodiment of the present invention, presented to the user, for providing recommendations to the user based on aggregate data from other program users. In this embodiment, a user is presented with testimonial examples of partner combinations that have yielded user weight loss success. In another embodiment, user data may be combined with aggregate user data to recommend partners, foods, activities, products, and techniques related to the program. In various embodiments, the administrator of the program may be agnostic as to the preferred or ideal methods of users for achieving physiological target conditions. Indeed, different solutions may work better for different groups and types of individuals. The administrator is in a position to accumulate data concerning the particular methods employed by users to more toward their physiological target conditions. Accordingly, embodiments of the present invention utilize this data to identify methods, types of programs, and strategies that are likely to be successful for individuals having the circumstances of a particular class of user. In particular, FIG. 52 illustrates how this data may be utilized. In one embodiment, partners may be chosen and displayed based on actual program success for a customized class of users, e.g., other members with similar demographics. In addition, partners may be chosen and displayed based on user recommendations. For example, a 30 yr. old male wanting to loose 20 lbs. may be shown the partners that were most successful and/or best rated for other 30 yr. old males wanting to loose approximately the same amount of weight. We discuss such an embodiment in further detail in connection with FIG. 58 below.

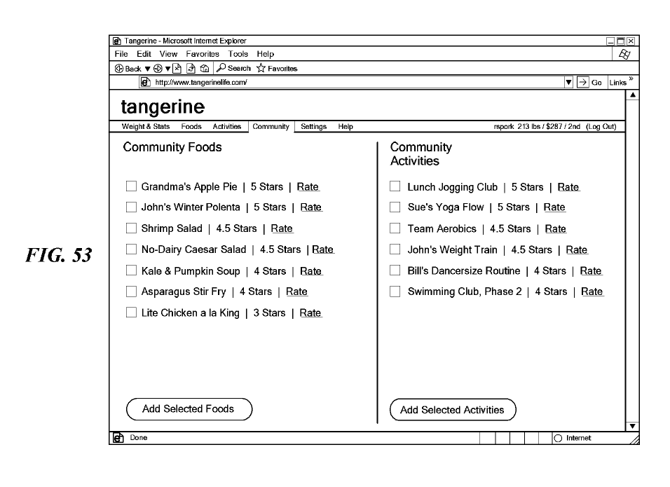

FIG. 53 is a representation of a web page, in accordance with an embodiment of the present invention, presented to the user, for showing foods and activities that may be selected, rated, and shared by the user among a community of users participating in the program. In this embodiment, using a web page, the user can view and rate community foods and activities. Similarly, the user can add community foods and activities to the user's food and activity logs.

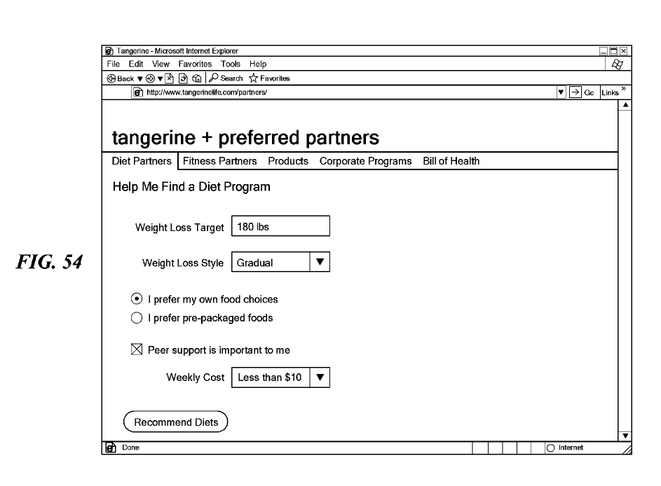

FIG. 54 is a representation of a web page, in accordance with an embodiment of the present invention, presented to the user, for providing options, for selecting a partner, that may be customized for the user taking into account patterns of similar users. In this embodiment, using a web page, the user can enter diet and exercise preferences as well as budgetary constraints. An administrator of the program or alternatively computer processes can use this information along with data from other users and third parties to recommend specific diet and exercise programs for the user.

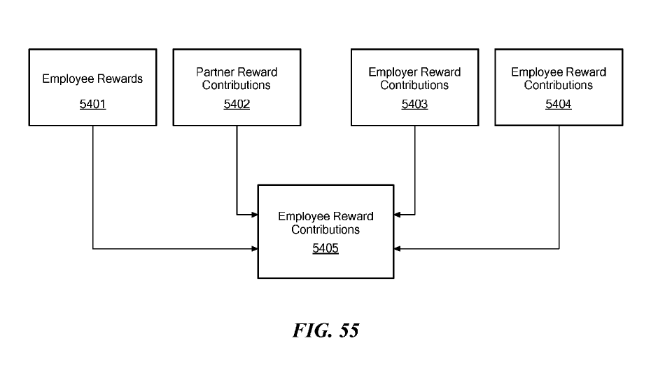

FIG. 55 is a block diagram, in accordance with an embodiment of the present invention, showing potential sources of user rewards as coming not only from contributions by an entity that is an employer, for example, but also from partners participating in the program and also from other users. In this embodiment, a reward pool is created from one or more of the following: employer, employee, partner, non-profit, or government entity. In this embodiment, rewards may be paid out in the form of points, cash, checks, debit cards, merchandise, travel, Health Savings Accounts (HSAs), Health Reimbursement Accounts (HRAs), or reduction in health insurance premiums.

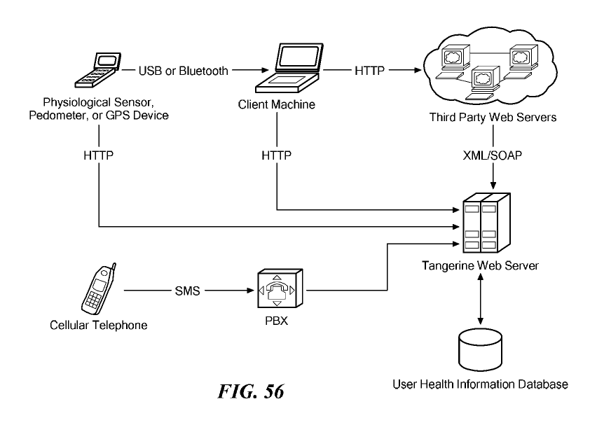

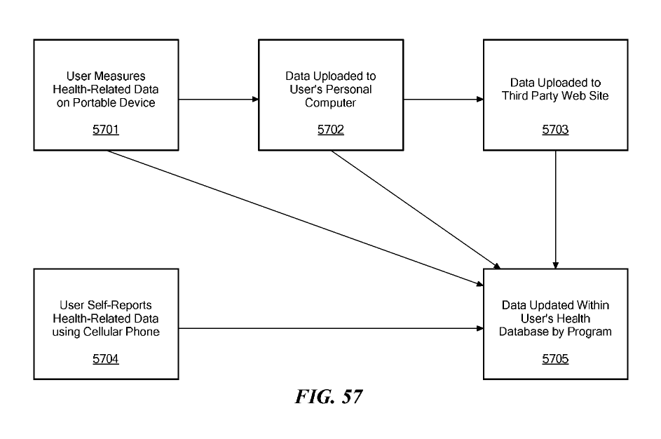

FIG. 56 is a block diagram showing potential sources for collecting and/or entering a user's physiological measurements and FIG. 57 illustrates how the data may be utilized in accordance with an embodiment of the present invention. In one embodiment, the user may enter the measurements through a cellular telephone (process 5704) and send the data, e.g., via SMS and a telephone network, to the program's web server. Alternatively, or in addition, the user may collect measurements using a small, wearable device (e.g., physiological sensor, pedometer, GPS device) (process 5701) and may send the data, e.g., via USB or Bluetooth, to a user's computer (process 5702). The computer may then send the data to third party web servers (process 5703), which may send the information to the program's web server or the computer may send the data directly to the program's web server. The information from the web server may then be stored in a user health information database in process 5705.

FIG. 58 is a block diagram of logical flow associated with the selection and recommendation of health resources offered to a user in accordance with an embodiment of the present invention. In one embodiment, the various health resource information may be entered by third parties in process 5801. The entity or its representatives may select which health resources it determines will be potentially available to its members or users in process 5802. The health resources may be recommended to the users by the program in process 5803. The users may rate the health resources (process 5804) and/or the program may measure the effectiveness of the health resource based on the actual success of the users (process 5805). Based on the ratings and/or the actual effectiveness of the health resource, recommendation rules may be established in process 5806, e.g., for a customized class of users such as other members with similar demographics. The recommendation rules may be used to display the best rated and/or most effective health resources to users in process 5803.

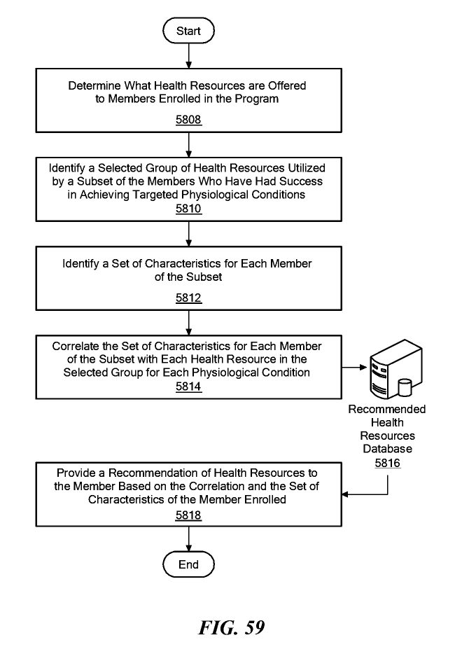

FIG. 59 is a block diagram showing the process of FIG. 58. In particular, in formulating a recommendation of a health resource to a member enrolled in a program, a process 5808 determines globally what health resources are offered to members enrolled in the program. Among these health resources, a process 5810 identifies a selected group of health resources utilized by a subset of the members who have had success in achieving targeted physiological conditions. A process 5812 also identifies a set of characteristics for each member of the subset; the set of characteristics for each member may include at least one characteristic selected from the group including gender, age, weight, and geographic location. In process 5814, the set of characteristics for each member of the subset is correlated with each health resource in the selected group for each physiological condition. These processes thus produce data showing with some granularity, based on member characteristics (such as gender, age, weight, and geographic location), what health resources have been associated with members who have had success in achieving their physiological targets; in process 5816, such data are stored in a recommended health resources database. In formulating a recommendation for a particular member, therefore, a process 5818 uses characteristics (such as gender, age, weight, and geographic location) of the particular member to access from such data, those health resources associated with members achieving their physiological targets who have one or more characteristics similar to one or more characteristics of the particular member.

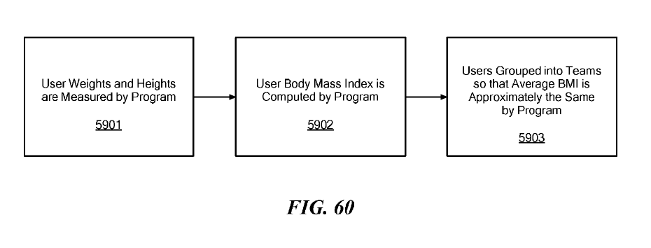

FIG. 60 is a block diagram of logical flow associated with grouping users into balanced teams in accordance with an embodiment of the present invention. In one embodiment, teams may be assigned based on a random selection of users so that each of the teams has approximately the same average BMI. In process 5901, the users weights and heights are measure or collected by the program. Each users' BMI is then computed by the program in process 5902. The program then selects or groups the users into teams based on the collective BMI of the users in process 5903.

It will be further understood by one of ordinary skill in the art that other modifications can be made without departing from the spirit and the scope of the invention, as set forth in the claims below.

What is claimed is:

1. A method for improving the health status of members of an entity by using a program in which members may enroll, the method comprising:
notifying the members of a pool of rewards available to those who enroll in the program;
for each enrolled member,
establishing a target physiological condition, and obtaining successive physiological measurements related to the condition over a period of time,
storing in a digital computer system the target physiological condition and the successive physiological measurements, wherein such measurements are obtained under personal supervision at a set of locations remote from the digital computer system and are provided to the computer system over a network, wherein at least some of the locations are under the control of an organization that is a measurement partner of the program; and
using a computer process to determine and store a score of such member's proximity to the target physiological condition based on each such measurement; and
granting an award from the pool to one or more enrolled members based on their scores at the end of the period.

2. A method according to claim 1, wherein at least some of such measurements are provided to the computer system via entries made in web pages over the network.

3. A method according to claim 1, wherein at least some of the measurements are collected using a small, wearable device.

4. A method according to claim 1, wherein at least some of the measurements are provided to the computer system via a cellular telephone.

5. A method according to claim 1, wherein the measurement partner is enrolled in the program by a process using web-page data entry over the network.

6. A method according to claim 1, wherein the entity is enrolled in the program by using web-page data entry, so as to be able to offer enrollment to members of the entity.

7. A method according to claim 1, wherein there exists a plurality of groups of enrolled members, further comprising:
determining a collective score for each of the plurality of groups based on the scores of enrolled members in each group, and
granting an award from the pool to one or more of the groups based on the scores of the groups.

8. A method according to claim 7, wherein the entity is a collection of organizations and each group is a distinct set of one or more organizations.

9. A method according to claim 1, wherein storing in a digital computer system the target physiological condition and the successive physiological measurements includes storing a combination of at least two of: the member's weight, blood pressure, body mass index, pulse rate, glycemic index, cholesterol level, levels of expression of designated genes, and ratio of body composition to fat percentage.

10. A method according to claim 1, wherein the entity includes a business entity.

11. A method according to claim 10, wherein the member is an employee of the business entity.

12. A method for improving the health status of members of an entity by using a program in which members may enroll, the method comprising:
notifying the members of a pool of rewards available to those who enroll in the program;
for a set including at least some of the members who enroll, causing each member of the set to be associated with one of a plurality of teams;
for each enrolled member,
establishing a target physiological condition, and obtaining successive physiological measurements related to the condition over a period of time,
storing in a digital computer system the target physiological condition and the successive physiological measurements, wherein such measurements are obtained at a set of locations remote from the digital computer system and are provided to the computer system over a network, wherein at least some of the locations are under the control of an organization that is a measurement partner of the program and wherein the measurement partner is enrolled in the program by a process using web-page data entry over the network; and
using a computer process to determine and store a score of such member's proximity to the target physiological condition based on each such measurement; and
determining a collective score for each of the plurality of teams based on the scores of enrolled members on each team, and
granting an award from the pool to one or more of the teams based on the scores of the teams.

13. A method according to claim 12, wherein causing each member of the set to be associated with one of a plurality of teams includes permitting each such member to voluntarily join one of the teams.

14. A method according to claim 12, wherein the entity is a collection of organizations, and causing each member of the set to be associated with one of a plurality of teams includes establishing a separate team for each organization, each team comprised of enrolled members from a single organization of the collection, so that the organizations compete with one another as teams.

15. A method according to claim 12, further comprising, for each enrolled member, making available to such member over a network in communication with the digital computer, information showing the member's progress toward such member's physiological target and, if applicable, competitive performance data for a team with respect to which such member is associated.

16. A method according to claim 12, wherein causing each member of the set to be associated with one of a plurality of teams includes balancing the teams so that an average of one or more characteristics of the members in each team is approximately equal.

17. A method according to claim 16, wherein balancing the teams includes calculating a body mass index for each member of the set and selecting each team so that the average body mass index for each team is approximately equal.

18. A method for improving the health status of members of an entity by using a program in which members may enroll, the method comprising:
notifying the members of a pool of rewards available to those who enroll in the program;
for each enrolled member,
establishing a target physiological condition, and obtaining successive physiological measurements related to the condition over a period of time,
storing in a digital computer system the target physiological condition and the successive physiological measurements, and
using a computer process to determine and store a score of such member's proximity to the target physiological condition based on each such measurement;
using stored data aggregated with respect to a selected class of enrolled members to identify a strategy successfully employed by members of the class to facilitate achieving their respective target physiological conditions, and communicating over a network in communication with the digital computer system a recommendation of the strategy to relevant enrolled members; and
granting an award from the pool to one or more enrolled members based on their scores at the end of the period.

19. A method for improving the health status of members of an entity by using a program in which members may enroll, the method comprising:
notifying the members of a pool of rewards available to those who enroll in the program;
for each enrolled member,
establishing a target physiological condition, and obtaining successive physiological measurements related to the condition over a period of time,
storing in a digital computer system the target physiological condition and the successive physiological measurements, and
using a computer process to determine and store a score of such member's proximity to the target physiological condition based on each such measurement;
communicating over the network to such enrolled member such member's progress toward the target physiological condition and, optionally, such member's behavior, and also optionally advertisements that are deemed pertinent to such member based on data pertinent to such member; and
granting an award from the pool to one or more enrolled members based on their scores at the end of the period.

20. A method according to claim 19, wherein obtaining successive physiological measurements includes collecting the measurements using a small, wearable device.

21. A method according to claim 20, wherein the small, wearable device includes a pedometer, a GPS device, a physiological sensor, or a combination thereof.

22. A method according to claim 19, wherein obtaining successive physiological measurements includes providing the measurements via a cellular telephone.

23. A method for improving the health status of a member enrolled in a program, the program providing a pool of rewards available to the member, the method comprising:
determining what health resources are offered to the members enrolled in the program;
identifying, among the health resources offered, a selected group of health resources utilized by a subset of the members who have had success in achieving targeted physiological conditions;
identifying a set of characteristics for each member of the subset;
correlating the set of characteristics for each member of the subset with each health resource in the selected group for each physiological condition; and
providing a recommendation of one or more health resources to the member based on the correlation and the set of characteristics of the member enrolled.

24. A method according to claim 23, wherein the set of characteristics for each member includes at least one characteristic selected from the group including gender, age, weight, and geographic location

25. A method according to claim 23, further comprising:
providing user recommendations of health resources that the members of the subset have had success in using to achieve targeted physiological conditions, wherein correlating the identifying characteristics for each member of the subset with each health resource in the selected group includes the identified selected group of health resources and the user recommendation of health resources.

26. A method according to claim 23, wherein determining what health resources are offered to the members enrolled in the program includes determining what to charge the health resource to be included as a resource based on success of the members in achieving targeted physiological conditions using the health resource.
